SEQUENCE LISTING (3 sequences)

SEQ ID NO: 1 - DNA, 19 bp, Artificial Sequence
  shRNA for targeting ST6GALNAC5
cataagcaactcaacaata

SEQ ID NO: 2 - DNA, 18 bp, Artificial Sequence
  shRNA targeting ST6GALNAC5
agcacatctccactgact

SEQ ID NO: 3 - DNA, 19 bp, Artificial Sequence
  target sequence for HB-EGF
ggtatgctgtcatggtcct

(19) United States
(12) Patent Application Publication
(10) Pub. No.: US 2010/0029748 A1
(43) Pub. Date: Feb. 4, 2010

(54) Metastasis Promoting Genes and Proteins

(75) Inventors: Joan Massague, New York, NY (US); Paula Bos, New York, NY (US)

Correspondence Address: Larson & Anderson, LLC;re: MSK, P. O. BOX 4928, DILLON, CO, 80435-4928, US

(73) Assignee: SLOAN-KETTERING INSTITUTE FOR CANCER RESEARCH, New York, NY (US)

(21) Appl. No.: 12/535,583

(22) Filed: Aug. 4, 2009

Related U.S. Application Data
(60) Provisional application No. 61/137,886, filed on Aug. 4, 2008.

(51) Int. Cl. A61K 31/7105; C12Q 1/02; C12Q 1/68

(52) U.S. Cl. 514/44 A; 435/29; 435/6

(57) ABSTRACT

Two sets of genes and their encoded proteins, one set of 17 genes/proteins and one set of 18 genes/proteins that can be used in predicting the risk of cancer metastasis to the brain, and as a screening assay to identify the suitable treatments for brain metastases. Genes/proteins within the sets that are found to be differentially expressed relative to a control value are suitable targets for therapy.

### STATEMENT OF RELATED CASES

This application claim the benefit of U.S. Provisional Application No. 61/137,886 filed Aug. 4, 2008, which application is incorporated herein by reference.

### STATEMENT OF GRANT SUPPORT

The invention described herein was developed in part with support from Grant No. U54 CA126518 from the National Institutes of Health. The government of the United States has certain rights in this invention.

### BACKGROUND OF THE INVENTION

Brain metastasis affects an estimated 10% of cancer patients with disseminated disease¹⁻⁴ Even small lesions can cause neurologic disability, and the median survival of patients with a diagnosis of brain metastasis is short-less than one year with surgery or radiotherapy. Lung, breast, melanoma, renal, and colon cancers, in this order of decreasing frequency, account for a majority of cases². Recent progress in the treatment of cancer has exposed brain metastasis as a growing problem because of its resistance to treatments that are otherwise effective against systemic disease³.

Brain metastasis has singular features that reflect the adaptation of cancer cells to the unique microenvironment of this organ. The brain parenchyma is very compact, lacks lymphatic drainage, and contains a dense microvascular network whose capillary walls constitute the blood-brain barrier (BBB)^{1,4}. The BBB consists of a continuous, non-fenestrated endothelium with tight junctions, no pinocytic activity, and high electrical resistance. It is surrounded by astrocytic foot processes, pericytes and a joint basal lamina, forming a barrier between blood and the cerebrospinal fluid that maintains the brain as an immunologically privileged site. Thus, brain metastasis requires circulating cancer cells to break through the BBB and subsequently infiltrate and colonize through the brain parenchyma. In addition to posing an obstacle for the entry of circulating tumor cells into the brain, the BBB also restricts the entry of therapeutic agents once brain metastases have developed⁵. The molecular determinants of brain metastasis and its prognostic factors remain unknown, although attention has been drawn to the fact that pulmonary metastases are commonly present when brain metastases are first diagnosed²

The current dearth of knowledge about the mechanisms of brain metastasis is recognized as a major obstacle to making progress against this condition^{3,5}. To address this problem, we used an integrated approach combining molecular, clinical, pharmacological, and functional evidence to identify genes and functions that mediate brain metastasis in breast cancer.

### SUMMARY OF THE INVENTION

Comparison of mRNA/protein levels in normal and disease tissue (for example cancerous tissue) can reveal a very large number of differences between the two types of samples. However, not all of the differences are unique to the disease condition, and not all are relevant of the existence of a disease condition. The present invention provides two sets of limited numbers of genes that are indicative of brain metastasis in cancer patients, particularly breast cancer patients, and which can be used in diagnostic testing and for selection of and as targets for therapeutic treatments to reduce brain metastases. The invention further provides a method for treatment of brain metastases in which an assay is conducted and the results are used to select a treatment based on inhibiting or enhancing expression of one or more differentially expressed genes.

### BRIEF DESCRIPTION OF THE FIGURES

FIGS. 1 A-D illustrate isolation and characterization of brain metastatic variants.

FIGS. 2 A-D are Kaplan Meier curves for brain metastasis-free survival in different patient cohorts.

FIG. 3A shows a schematic of the in vitro assay used to determine the migration of tumor cells through an in vitro BBB model.

FIG. 3B shows result of an albumin permeability analysis to determine the tightness of the endothelial layer. Absorbance at 620 nm is shown relative to an empty tissue culture insert. Data are the average of triplicate determinations±S.D.

FIG. 3C shows results for tests on in vitro BBB transmigration activity of the indicated cell lines and gene knockdown derivatives. Number of transmigrated cells relative to the parental cell lines is plotted. n=6-20. p-values are calculated using a one-tailed unpaired t-test.

FIG. 3D shows Kaplan-Meier curves for brain metastasis-free survival of mice injected with CN34-BrM2c (left panel) or MDA231-BrM2a (right panel) cells expressing shRNA vector control, or shRNA targeting COX-2.

FIG. 3E shows Kaplan-Meier curves for brain metastasis-free survival of mice injected with CN34-BrM2c (left panel) or MDA231-BrM2a cells (right panel) and treated with cetuximab or vehicle. p-values are calculated by log-rank (Mantel-Cox) test.

FIG. 4A shows quantification of SNA staining in 10 representative fields of the tumors using Metamorph software.

FIG. 4B shows distribution of SNA staining intensity in 6 brain, 4 lung, and 3 liver metastases resected from breast cancer patients.

FIG. 5A shows adhesion of the indicated CN34, CN34-BrM2 and ST6GALNAC5 derivatives to a monolayer of primary brain microvascular endothelial cells.

FIG. 5B shows in vitro BBB transmigration activity of the indicated cell lines.

FIG. 5C shows Kaplan-Meier curves for brain metastasis-free survival of mice injected with CN34-BrM2a cells expressing a hairpin targeting ST6GALNAC5 compared to control cells expressing the empty vector. p-values are calculated by log-rank (Mantel-Cox) test

FIG. 5D shows a schematic model of organ-specific metastatic extravasation of breast cancer cells. Extravasation into the bone marrow is a relatively permissive process owing to the fenestrated endothelium lining the sinusoid capillaries. Extravasation into the pulmonary or brain parenchyma requires specific functions for breaching the endothelial barriers of these tissues. Shared mediators of extravasation include COX-2, EGFR ligands such as epiregulin and HB-EGF, and others. However, the stringent nature of the BBB requires additional mediators of cancer cell extravasation including, among others, the brain-specific sialyltransferase ST6GalNac5 whose activity enhances cancer cell interactions with the brain endothelium.

FIGS. 6A-D show Kaplan-Meier curves for brain metastasis-free survival in ER-negative tumors from the two independent validation datasets (EMC-204 and NKI-295); Kaplan-Meier survival curves for bone, and liver metastasis-free survival in all EMC and NKI tumors (EMC-286, EMC-204 and NKI-295). Lymph node metastasis-free survival is shown for the NKI-295 dataset, which was the only cohort with both lymph node positive and negative tumors; and Kaplan-Meier curves for lung metastasis-free survival using the LMS or the BrMS as classifier.

FIGS. 7A and B show Western immunoblot analysis of COX-2 protein in the indicated parental, BrM2c and COX-2 knockdown cell lines.

FIGS. 7C and D show qRT-PCR analysis of mRNA levels for EREG, HB-EGF and ST6GALNAC5 in the indicated cell lines.

FIG. 8 shows an Oncomine Cancer Microarray database analysis of ST6GALNAC5 expression in human tissues 39. p-value was calculated using Student's t-test.

### DESCRIPTION OF THE INVENTION

The present invention provides two sets of genes and their encoded proteins, one set of 17 genes/proteins and one set of 18 genes/proteins that can be used in predicting the risk of cancer metastasis to the brain, and as a screening assay to identify the suitable treatments for brain metastases. Genes/proteins within the sets that are found to be differentially expressed relative to a control value are suitable targets for therapy. The term “genes/proteins” is used in the specification and claims of this application to reflect that expression levels may be determined either by measurement of mRNA levels or protein levels.

To determine the first set of genes, cells cultured from two different pleural effusion samples of breast cancer patients (one was ER+, and the other ER−) were introduced into mice. Brain tumors that developed were harvested and cells were cultured. These cells were introduced into mice for a second round of in vivo selection and the resulting brain tumors were harvested. These cells are referred to herein collectively as BrM2 cells. Evaluation of expression levels of multiple proteins led to the surprising finding that 50 genes were differentially expressed in BrM2 regardless of the origin/type of the sample. These 50 genes were then cross-referenced with the gene-expression data sets for a cohort of breast cancer patients who had exhibited brain metastases to obtain a reduced set of genes. After excluding genes that had high variance or discrepancy between the two original cell lines, the 17 gene set shown in Table 1 was obtained. This gene set is referred to herein as the brain metastasis gene expression signature (BrMS). Individually, or in combination, these genes' expression, or the levels of the protein they code for, can be modified, through inhibitory or stimulatory agents, for the treatment of brain metastasis. Another embodiment of the present invention is a determining which gene(s) or protein(s) are elevated or decreased in the brain metastasis of certain cancer patient, these genes selected from the list of the 17 genes that are listed in Table 1, with respect to a normal control sample and choosing a therapy that is directed towards normalizing the levels of such gene(s) or protein(s) for the treatment of brain metastasis.

The second set of genes is a set of genes that is over expressed in the BrM2 cells but that are not part of the BrMS. These genes were selected from among the genes showing at least a 3-fold difference in expression relative to the parental line, and were not part of a previously disclosed lung-metastasis signature or bone metastasis signature. The resulting set of 18 genes is shown in Table 2. Investigation of members of this set of genes supported a role in supporting the occurrence of brain metastases through extravasation through the blood brain barrier (BBB). Another embodiment of the present invention is a determining which gene(s) or protein(s) are elevated or decreased in the brain metastasis of certain cancer patient, these genes selected from the list of the 18 genes that are listed in Table 2, with respect to a normal control sample and choosing a therapy that is directed towards normalizing the levels of such gene(s) or protein(s) for the treatment of brain metastasis.

We further investigated the gene encoding ST6GALNAC5 which is a member of the second set of genes because it was unique among the proteins encoded by this gene set in providing a sialylation function. Based on this investigation, it is another object of the present invention to determine the gene expression levels, or the protein expression levels of ST6GALNAC5, in the lung or brain metastatic lesions of a cancer patient. If those levels are higher than certain threshold considered normal, then this cancer patient can benefit from being provided a therapy to lower the level or activity of ST6GALNAC5.

The genes identified in Tables 1 and 2 can be used in a method of predicting the likelihood of brain metastases in a patient. Even if metastasis has not been observed in a patient, a sample such as a plural effusion from a patient previously treated for cancer, particularly breast cancer, can be tested to determine a gene expression signature using members of either or both gene sets, either individually or in combination. Identification of a risk of metastasis can be used in the selection of appropriate therapy, including both the rigorousness of the therapy as well as the selection of a therapy targeted to a specific gene that is differentially expressed in the sample from the patient relative to pre-defined control values.

The invention also provides a kit for the analysis of expression levels for the genes in Table 1 and/or 2. For example, a kit may include an antibody array or a DNA array (for example an Affymetrix™ chip) for the proteins/genes of interest. Thus, the kit which is specific for the method of the invention comprises reagents for assessing expression levels, wherein the reagents consist of compounds specific for one or more of the genes/proteins of Table 1 and/or Table 2.

The two gene sets can also be used in connection with “treating” brain metastases in a patient in need of such treatment. The term “treating” as used herein encompasses providing the therapy as described with the goal of delaying the onset of or reducing the severity of brain metastases whether or not this goal is achieved in an individual patient, and can be applied to patients with a perceived risk for but no observed metastases or to patients with observed brain metastases.

The first step in the method for treating brain metastases is determining the expression level of at least one of the genes, and optionally all of the genes from Table 1 and/or Table 2 in a patient sample, for example a pleural effusion, tumor biopsy, brain metastasis biopsy, circulating tumor cells from blood samples, or cerebrospinal fluid. From this expression level, genes/proteins which are differentially expressed are identified as one or more therapeutic targets, and a therapeutic composition is administered to normalize (reduce or increase to more closely approximate the control level) the level of the one or more therapeutic targets.

Therapeutic agents which can reduce the level of an overexpressed gene/protein include those generally known in the art, such as antibodies, antisense, shRNA, siRNA at the like. Administration may be by injection, for example, intravenous, intrathecal, intramuscular or subcutaneously, intranasal, and oral. In many cases, and particularly in the case of already detected brain metastases the use of a therapeutic composition which either passes through the blood brain barrier or administration that avoids this requirement (for example intrathecal or intranasal) is desirable. Combinations of treatment that provide both systemic and brain availability of the therapeutic may also be used.

Non-limiting examples of agents suitable for formulation with therapeutic agents include: P-glycoprotein inhibitors (such as Pluronic P85), which can enhance entry of drugs into the CNS (Jolliet-Riant and Tillement, 1999, Fundam. Clin. Pharmacol., 13, 16-26); biodegradable polymers, such as poly(DL-lactide-coglycolide) microspheres for sustained release delivery after intracerebral implantation (Emerich, D F et al, 1999, Cell Transplant, 8, 47-58) (Alkermes, Inc. Cambridge, Mass.); and loaded nanoparticles, such as those made of polybutylcyanoacrylate, which can deliver drugs across the BBB and can alter neuronal uptake mechanisms (Prog Neuropsychopharmacol Biol Psychiatry, 23, 941-949, 1999). See also, US 2005/0202075, WO2005/003350, US2005/042646, and GB2415961.

In the methods and kits of the invention, the invention can make use of any gene/protein from Table 1 or 2, all of the genes/proteins from Table 1 and/or Table 2, or any intermediate number of genes/proteins from Table 1 and/or Table 2. For example, the methods may make determinations for any 5 or 10 genes/proteins from Table 1 and/or 2, and this is suitably done using a kit containing compounds specific for just these 5 or 10 genes/proteins.

Experimental Results and Discussion

### Isolation of Brain Metastatic Cells

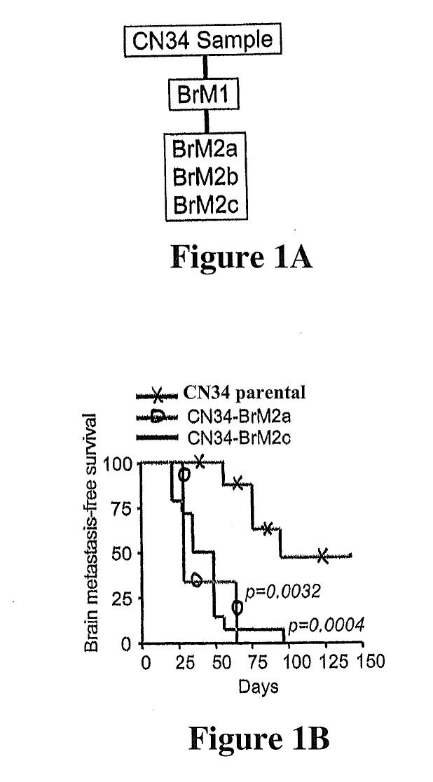

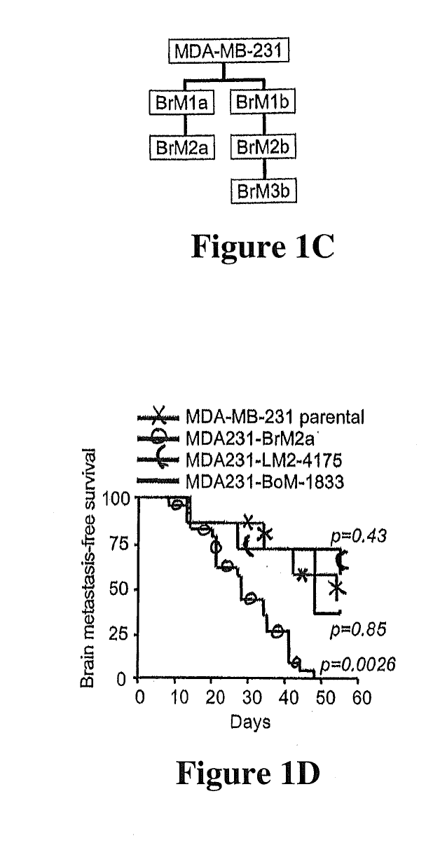

Pleural effusions from patients with metastatic breast cancer contain malignant cells with different organ tropisms⁶, This heterogeneity is thought to reflect the presence of cancer cells that originally colonized different organs and were later re-dispersed and intermixed as the disease progressed. To isolate brain metastatic cells from such mixed populations, we established cultures of pleural malignant cells from a breast cancer patient who was treated at Memorial Sloan-Kettering Cancer Center (CN34 sample) (FIG. 1A). These cells were estrogen receptor-negative (ER−) (data not shown), although the original breast tumor had been diagnosed as ER+. We also used MDA-MB-231 (MDA231 for brevity), an ER− cell line that was established from the pleural fluid of a breast cancer patient⁷ and previously used for the isolation of bone and lung metastatic cells^{8,9} (FIG. 1C). CN34 and MDA231 cells were inoculated into the arterial circulation of immunodeficient female mice to allow distribution to all organs. Growing tumors were tracked by bioluminescence imaging of a stably transduced GFP-luciferase fusion protein. For example, bioluminescence imaging showed brain and leptomeningeal metastasis by CN34-BrM2c cells in at least some mice. Mice that developed tumors in the head were examined by magnetic resonance imaging (MRI) and ex vivo whole-brain bioluminescence imaging to verify that the lesions were located in the brain parenchyma. After harvesting and expanding these lesions in culture, the resulting cell populations (BrM1) were subjected to a second round of in vivo selection and extraction, yielding BrM2 cell populations. BrM2 lines derived from CN34 and from MDA231 had a significant gain in brain metastatic activity compared to the respective parental populations, and compared also to lung metastatic and bone metastatic MDA231 derivatives (FIGS. 1B and D). Cells from a third round of in vivo selection (BrM3) showed no further gain in metastatic activity.

The CN34-BrM2 and MDA231-BrM2 cell lines generated brain lesions with full penetrance and similar phenotypes. As revealed by H&E staining, multifocal lesions were frequently located in the cerebrum, but also in the cerebellum and the brainstem. Meningeal metastases were frequently observed in the encephalic leptomeninges as well as the leptomeninges lining the spinal cord and optical nerve. This pattern is consistent with metastatic breast cancer being the tumor with the highest propensity to invade the leptomeninges¹⁰. Brain parenchyma lesions were diffuse and invasive, and were often located at the junction of the gray and white matter. Larger nodules developed hemorrhagic cores and edema visualized by MRI.

A pronounced astrogliosis occurred in the periphery of the tumors, as evidenced by immunostaining of tissue sections for the astrocyte intermediate filament marker glial fibrillary acidic protein (GFAP). Tumor cells express green fluorescent protein (GFP), and glial cells were stained with the glial marker GFAP (purple). BP, brain parenchyma. All these features are typical of brain metastasis in cancer patients^{4,11}

When inoculated into the mammary fat pad, CN34-BrM2 formed tumors that metastasized to brain in 42% (5/12) of the mice, demonstrating the ability of these cells to form brain metastases upon dissemination from the orthotopic site. Within 24 h of direct inoculation into the circulation, BrM2 cells could be found lodged in brain capillaries as single cells using DAPI staining of the nuclei indicating that brain metastases result from an ability of these cells to breach the BBB.

Genes Associated with Brain Metastasis

To identify genes whose expression is associated with brain metastatic activity, we conducted comparative genome-wide expression analysis of the parental cell lines versus their corresponding BrM derivatives. Using a 2.5-fold change, and p<0.05 as cut-off values, 271 genes (310 probe sets) were differentially expressed between the parental and brain-metastatic CN34 cell lines, and 179 genes (210 probe sets) between the parental and brain-metastatic MDA231 cell lines (Tables 3 and 4). Remarkably, 50 genes (54 probe sets) were shared between these two independent sets.

To determine whether the expression of these genes is linked to brain relapse in patients, we performed univariate analysis of the association of each differentially expressed gene with brain metastasis-free survival. We used gene-expression data sets corresponding to a cohort of 368 clinically annotated breast tumors (82 tumors from Memorial Sloan-Kettering Cancer Center, MSK-82 set; and, 286 from Erasmus Medical Center, EMC-286 set). The signal intensity of 31 probe sets had a significant correlation (p<0.05) with brain relapse in this cohort. Filtering genes with a large variance or discrepancy between the different cell lines reduced this list to 18 probe sets corresponding to 17 unique genes (Table 1). We termed this gene set the Brain Metastasis gene-expression Signature (BrMS). Thirteen BrMS genes were positively associated with brain metastasis in patients and in the cell lines, and four were negatively associated, establishing groups of putative mediators and suppressors of brain metastasis, respectively (Table 1).

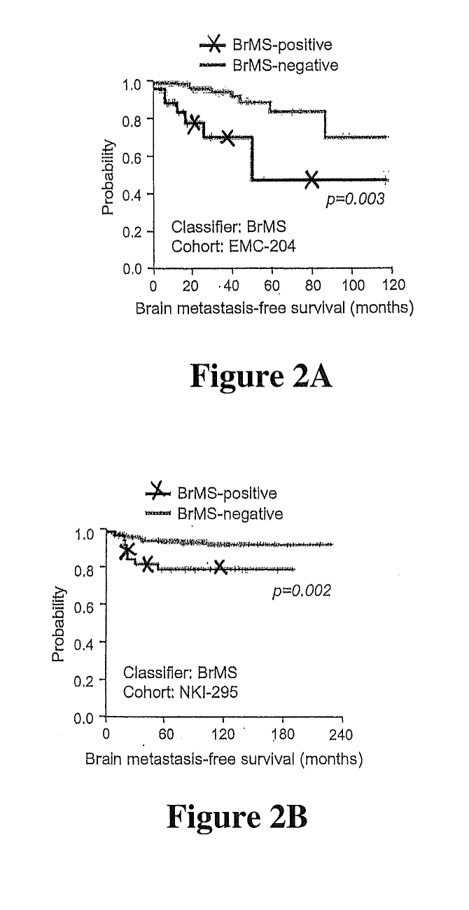

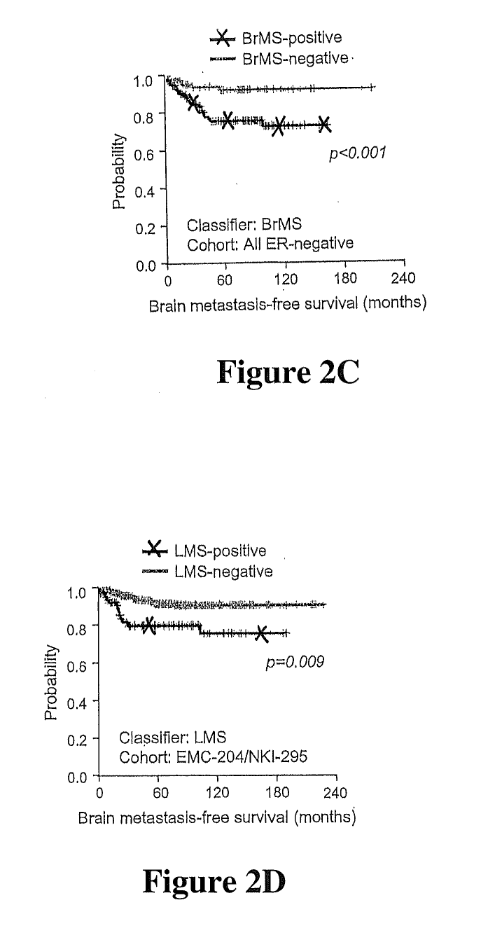

Unsupervised hierarchical clustering of the 368 tumors revealed a group of tumors with an expression pattern for these 17 genes resembling that of the BrM cell lines. We developed a classifier by training the BrMS gene set with the BrMS+ and BrMS− tumors in the 368-tumor cohort. This classifier was then tested on two independent datasets, including an additional 204 primary tumors from the Erasmus Medical Center (EMC-204 cohort), and a group of 295 primary tumors from the Netherlands Cancer Institute (NKI-295 cohort). Indeed, tumors that scored as BrMS+ were associated with brain relapse in both the EMC-204 (p=0.003) and the NKI-295 cohorts (p=0.002) (FIGS. 2A and B). As a control, we randomly generated ten sets of 500 genes each, which is a similar number to our initial gene list, and determined the correlation of the genes in each set with brain metastasis-free survival in the 368-tumor cohort. We then selected the significant genes, and tested these subsets as we did with the BrMS. These random gene sets yielded p values for brain relapse association ranging from 0.1 to 0.8. Thus, we concluded that the performance of the BrMS as a correlate of brain relapse was not due to chance.

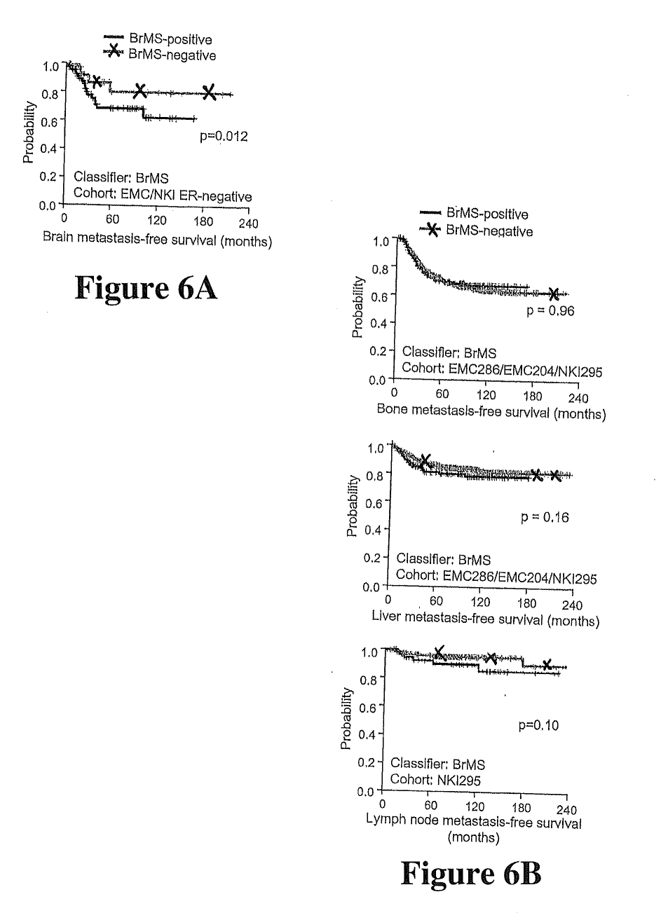

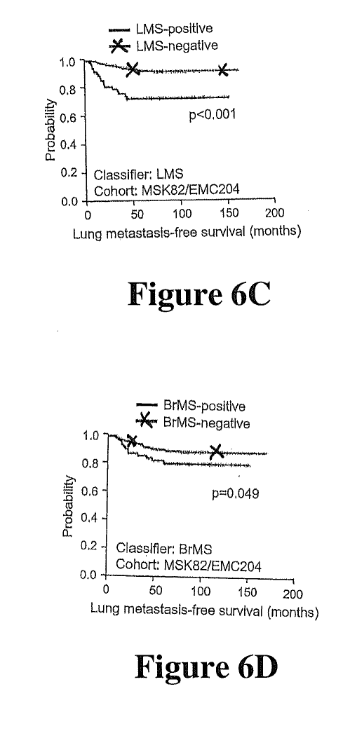

ER negative breast tumors relapse to the brain more frequently than do ER+tumors (Table 5). The association of BrMS+ status with brain relapse remained significant within the ER− subset of tumors from the combined EMC-204 and NKI-295 cohorts, (FIG. 6A) or from all four cohorts combined (FIG. 2C). The association of BrMS+ status with brain metastasis in ER+ tumors did not reach statistical significance (p=0.08 in all cohorts combined; data not shown).

A Link with Lung Metastasis

BrMS+ status in breast tumors was not linked with relapse to bones (p=0.96), liver (p=0.16) or lymph nodes (p=0.10) (FIG. 6B). Only one gene (MMP1) was shared between the BrMS and a bone metastasis gene set previously identified using the MDA231 system⁸. Surprisingly, however, six BrMS genes were shared with an 18-gene Lung Metastasis Signature (LMS) that was previously elucidated using a similar approach⁹. LMS+ status is associated with relapse to lung but not to bones, liver or lymph nodes (FIG. 6C; Ref.¹²). Interestingly, LMS+ status was significantly associated with relapse to brain (FIG. 2D), and BrMS+ status with relapse to lung (FIG. 6D). The association of BrMS with lung relapse (p=0.049) was weaker than with brain relapse (p=0.003). Similarly, the association of LMS with brain relapse (p=0.009) was weaker than with lung relapse (p<0.001).

Lung as a first site of relapse is linked to brain metastasis in breast cancer patients^{2,13}. Although the pattern of hematogenous dissemination of cancer cells has been considered as a possible explanation, the basis for the link between lung and brain metastasis has remained unknown. The present finding that the BrMS and the LMS share certain genes suggests a molecular basis for this link. The shared genes include the prostaglandin-synthesizing enzyme cyclooxygenase 2 (PTGS2/COX-2), which promotes breast cancer cell extravasation into the lung parenchyma 14; the matrix metalloproteinase collagenase-1 (MMP1), which is implicated in invasion and extravasation^{14,15}; angiopoietin-like 4 (ANGPTL-4), a gene induced by tumor-derived TGFbeta to disrupt lung capillary endothelial cell junctions for cancer cell extravasation¹⁶; latent TGFbeta-binding protein (LTBP1), which participates in TGFbeta activation¹⁷; the cytoskeletal component of lamellipodia fascin-1 (FSCN1), which is implicated in cancer cell migration¹⁸; and the putative metastasis suppressor RARPES3¹⁹. Additionally both signatures include an epidermal growth factor receptor (EGFR) ligand, heparin-binding EGF (HB-EGF) in the case of the BrMS, and epiregulin (EREG) in the LMS (Table 1; Ref.⁹). Of note, EREG was also upregulated in the CN34-BrM cells (Table 3), although it was not differentially expressed in the MDA231 cells. EREG cooperates with COX-2 and MMP1 in experimental lung metastasis¹⁴. These observations suggested that breast cancer cells seed the brain in part by resorting to functions that also mediate metastatic seeding of the lungs, but not the bones, liver, or lymph nodes.

### Mediators of Blood-Brain Barrier Breaching

Compared to extravasation through the endothelial lining of the lung capillaries, extravasation of cancer cells into the brain parenchyma is thought to be particularly challenging owing to the impediment of the BBB⁴. Given the overlap between the BrMS and LMS gene sets, we were interested in testing the hypothesis that breast cancer cells extravasate through the BBB by using a combination of functions provided by lung extravasation genes plus additional functions provided by genes that are uniquely involved in brain metastasis.

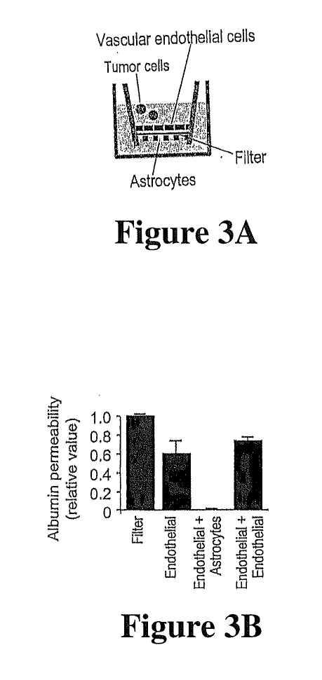

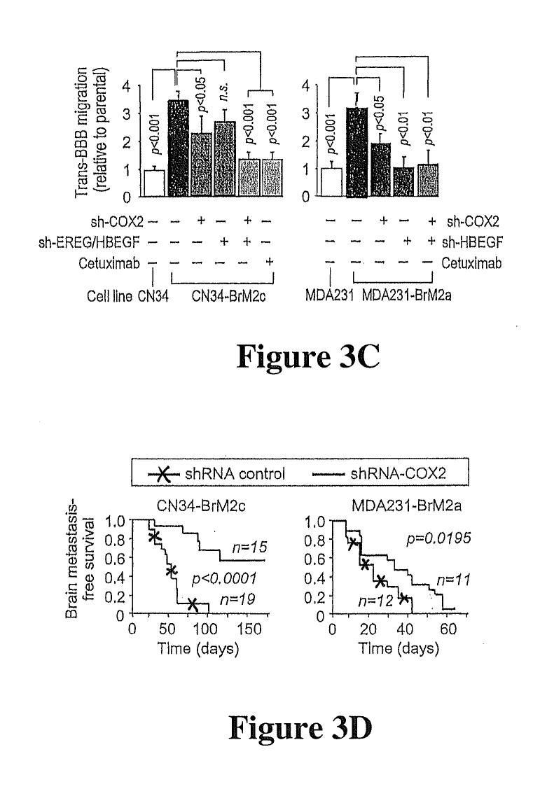

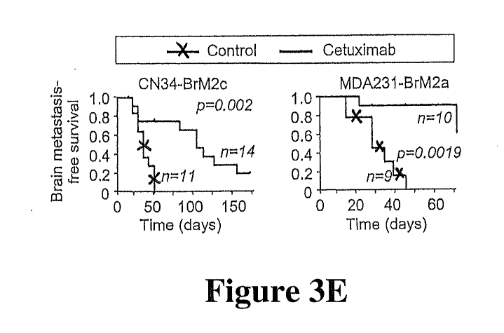

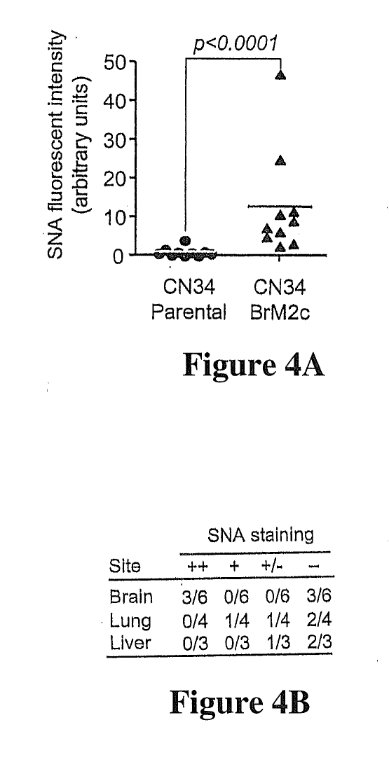

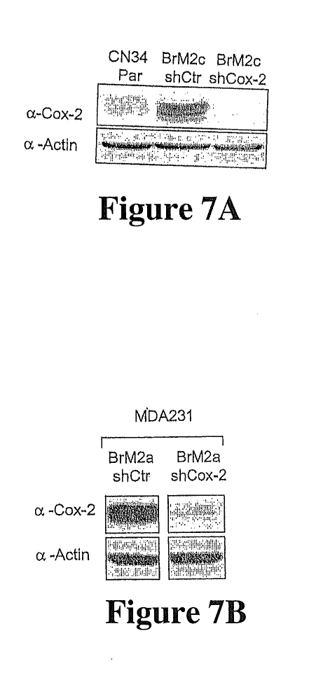

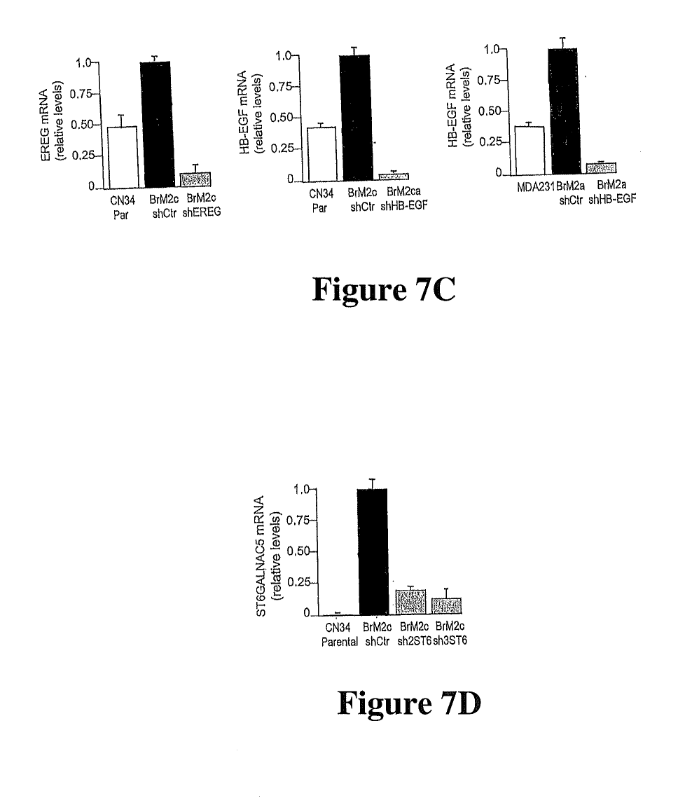

To test the first aspect of this hypothesis, we focused on COX-2 and EGFR ligands, which are present both in the BrMS and the LMS, and were previously implicated in extravasation of breast cancer cells into the lungs¹⁴. We used an in vitro BBB model that is based on human primary endothelial cells and astrocytes, plated on opposite sides of a porous membrane in a tissue culture insert (FIG. 3A)²⁰. After several days in culture, the endothelial monolayer in the upper side of the membrane differentiated into a tight barrier that resembles a BBB, as determined by its lack of permeability to albumin (FIG. 3B). Cancer cells in suspension were placed in the upper chamber of the inserts, and the BBB transmigration capacity of these cells was determined based on the proportion that migrated into the lower chamber. The CN34-BrM2 and MDA231-BrM2 lines were 3-4-fold more active than their parental lines (FIG. 3C). Knockdown of COX-2 expression using a previously validated short-hairpin RNA (shRNA) (Ref.¹⁴; FIGS. 7A and B) significantly inhibited BBB transmigration in both BrM2 lines (FIG. 3C). Knockdown of HB-EGF alone in the MDA231 BrM2 cells also significantly inhibited BBB transmigration (FIG. 3C). In contrast, a combination knockdown of HB-EGF and EREG in CN34-BrM2 cells did not show significant reduction in their ability to cross the BBB in vitro. Yet, addition of the therapeutic anti-human EGFR antibody cetuximab to the media markedly decreased the BBB transmigration activity of CN34 BrM2 cells (FIG. 3C), suggesting that CN34-BrM2 cells employ multiple mechanisms to activate the EGF receptor pathway whereas MDA231-BrM2 primarily rely on HB-EGF.

When injected into the arterial circulation of mice, COX-2 knockdown BrM2 cells showed lower brain metastatic activity compared to control BrM2 cells (FIG. 3D). Brain metastasis-free survival could also be prolonged by pre-treating BrM2-inoculated mice with cetuximab (FIG. 3E). Cetuximab is reported to recognize human but not murine EGFR²¹, arguing that its inhibitory effect on brain metastasis was due to EFGR blockade in the human cancer cells.

These results suggest that COX-2 and EGFR ligands in BrM2 cells mediate BBB transmigration and brain metastasis. Of note, the transmigration of MDA231 lung metastatic derivatives through a simple endothelial layer, and the lung metastatic activity of these cells in vivo can be suppressed with combined inhibition of COX-2 and EREG, but not by individually targeting these two activities¹⁴. By contrast, the ability to inhibit BBB transmigration and brain metastasis by individually targeting COX-2 or EGFR indicates a greater dependence of brain metastasis on these mediators.

### Brain Metastasis Specific Genes

To search for genes that specifically enhance cell passage through the BBB we considered the subset of BrMS genes that are not shared with the LMS. These genes include the extracellular matrix proteins laminin alpha 4 (LAMA4) and collagen type XIII α1 (COL13A1), the collagen-modifying enzyme PLOD2, the cytokine granulocyte colony-stimulating factor (CSF3), and others (Table 1). We additionally considered a subset of genes that were not part of the BrMS but were differentially overexpressed >3-fold in both the CN34-BrM2 and MDA231-BrM2 cell lines compared to the respective parental lines (Tables 3 and 4). After excluding genes that were also overexpressed in bone metastatic⁸ or lung metastatic MDA231 derivatives⁹ and histone genes, we arrived at a set of 18 candidates (Table 2). This set largely consists of cell-cell communication components (protocadherin-7 and connexin-43), secreted proteases (PRSS3/mesotrypsin and MMP3/stromelysin-1), protase inhibitors (serpin-2 and neuroserpin), G-protein regulators (Rho guanine nucleotide dissociation inhibitor ARHGDIB, Ran GTPase-activating protein GARNL4, and heteromeric G-protein inhibitor RGS2), inflammatory signaling components (interleukins IL1A and IL1B and the toll-like receptor TLR4), and the α-2,6 sialyltransferase ST6GALNAC5. The functions encoded by this group of genes are remarkably suggestive of roles in metastasis. Malignant cells from the primary tumor that stochastically express these genes might enjoy an added advantage only upon reaching the brain.

### A Role for Sialylation in Brain Metastasis

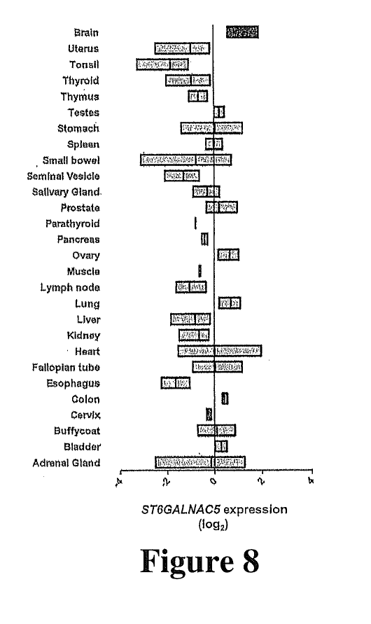

To investigate the specific role of this particular class of genes in brain metastasis and more specifically, in extravasation through the BBB, we chose to focus on ST6GALNAC5. This gene is primarily expressed in the forebrain and cerebellum in mice^{22,23}. In human, ST6GALNAC5 expression is also highest in the brain (FIG. 8), suggesting that brain metastatic breast cancer cells have co-opted the expression of a brain sialyltransferase. ST6GalNac5 is a transmembrane protein that catalyzes the formation of α-2,6 linkages between sialic acid and N-acetylgalactosamyl residues of cell surface gangliosides²². Sialylation of surface molecules has been implicated in the modulation of cell-cell interactions including interactions between invasive cancer cells and their microenvironment²⁴.

As the only member of its class in our list of selected genes, we wondered whether ST6GalNac5 plays a rate-limiting role in brain metastasis extravasation through this largely unexplored mode of cancer cell-endothelium interaction. Using qRT-PCR we confirmed that ST6GALNAC5 is highly expressed in CN34-BrM2 cells (>100-fold relative to the parental cell line), and MDA231-BrM2 cells (30±1 fold), as well as in two additional pleural-derived samples that were subjected to one cycle of selection in mice, CN37-BrM1 (95±23 fold) and CN41-BrM1 (72±12 fold). To verify that ST6GalNac5 activity results in the accumulation of α-2,6 sialyl groups in the tumor cells, we stained these cells with Sambucus nigra agglutinin (SNA), a lectin that specifically binds to α-2,6-linked sialyl groups²⁵. CN34-BrM2 cells monolayers showed strong and extensive SNA staining compared to parental CN34 cells. Profuse 2,6-linked sialyl staining with SNA was observed in mammary tumors formed by CN34-BrM2 cells but not in tumors formed by parental CN34 cells. Brain metastases generated by MDA231-BrM2 and CN34-BrM2 cells showed intense SNA staining compared to the surrounding brain parenchyma.

Of six brain metastasis samples obtained from different breast cancer patients, three contained areas that stained strongly with SNA, whereas lung or liver metastasis samples stained weakly if at all. We examined ST6GALNAC5 expression in an Affymetrix (U133A) gene expression dataset that included 13 brain metastases samples and 24 samples of metastasis to other sites (lung, bone, liver and ovary) from breast cancer patients. All these samples were ER− as defined by the intensity of the ESR1 probe. ST6GALNAC5 expression level approximated that of the BrM2 cell lines in 23% (3/12) of the brain metastases samples but in none of the metastases to other sites, a difference that was statistically significant (p=0.04, Fisher's Exact Test).

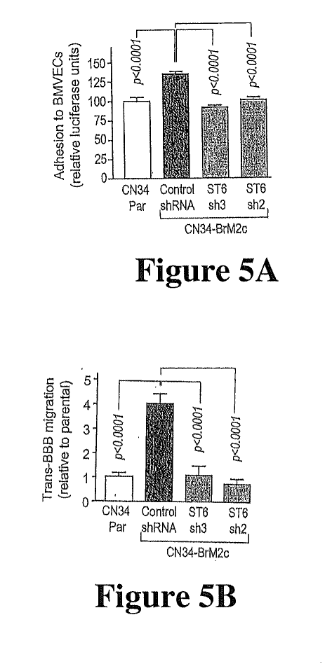

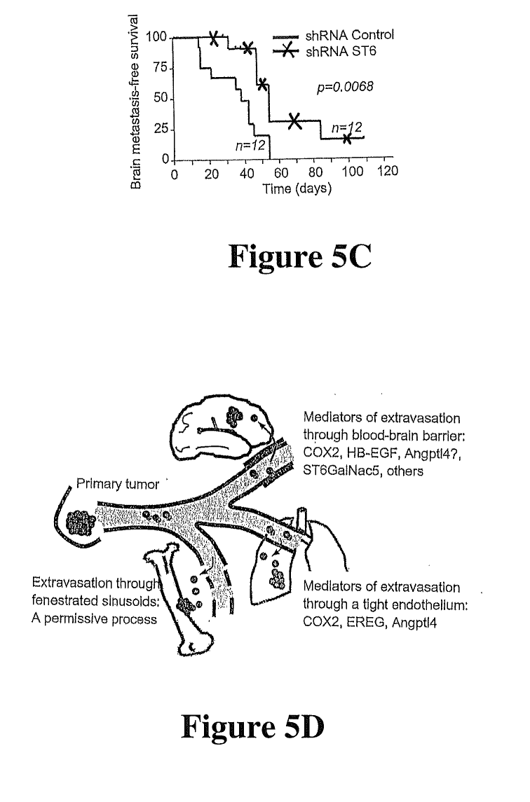

To test the role of ST6GalNac5 in tumor cell adhesion to endothelial cells, we compared the ability of parental and brain metastatic CN34 lines to adhere to monolayers of human primary brain endothelial cells. CN34-BrM2 cells were significantly more adhesive to these monolayers than were the parental CN34 line or two independent ST6GALNAC5-knockdown CN34-BrM2 derivatives (FIG. 5A). The knockdown of ST6GALNAC5 with two independent shRNAs strongly decreased the BBB transmigration activity of CN34-BrM2 cells (FIG. 5B). Moreover, the knockdown of ST6GALNAC5 significantly decreased the brain metastatic activity of CN34-BrM2 cells inoculated into the arterial circulation of mice (FIG. 5C; FIG. 7C). Collectively these results suggest that cell-cell interactions that depend on ST6GalNac5 are rate limiting for BBB extravasation by metastatic breast cancer cells.

### Deconstructing Brain Metastasis

The ability of disseminated cancer cells to colonize distant organs depends on the acquisition of functions that defeat the barriers imposed by particular organ microenvironments²⁶-29. In the present work we have identified genes whose expression enables circulating breast cancer cells to penetrate and colonize the brain. Many of these genes may not confer a selective advantage to cancer cells in the primary tumor microenvironment if the functions that they encode become critical to these cells only upon reaching the brain. As such, the expression of these genes would not be detectable in global transcriptomic analysis of primary tumor samples. The genes listed in Table 2 fall in this class, which we refer to as metastasis virulence genes³⁰.

Other mediators of organ-specific metastasis however are detectably expressed in primary tumors⁹. The BrMS genes in Table I fall in this class, which we refer to as metastasis progression genes. Their abundant expression in breast tumors may be a sign that these genes provide a selective advantage in the primary tumor besides providing a distinct advantage in brain metastasis. Among the BrMS genes that mediate BBB extravasation, COX-2 and EGFR ligands have been previously shown to also promote vascular assembly in mammary tumors¹⁴, while the BrMS gene ANGPTL4 is one of many TGFbeta target genes whose expression in breast tumors merely reflects the presence of TGFbeta activity in the tumor microenvironment without providing any discernable advantage. Yet, it enhances the extravasation of disseminating tumor in the lungs¹⁶. We surmise that ANGPTL4 may play a similar role in extravasation through the BBB.

The sharing of one-third of the genes between the BrMS and the LMS was an unexpected result, but one that provides an explanation for the long-standing clinical observation of a link between relapse to the lungs and to the brain^{2,13} (schematically summarized in FIG. 5D). The shared genes prominently include the extravasation mediators COX-2, ANGPTL4, MMP1 and EGFR ligands^{14,16}, as well as FSCN1 and LTBP1, which are implicated in tumor cell migration and TGFbeta regulation in the tumor microenvironment, respectively^{17,18}. The association of these genes with relapse to brain and lung, but not relapse to bone, liver or lymph nodes, suggests similarities in the specific requirements for cancer cell entry and colonization of brain and lung. The most apparent of these similarities is in the structure of the blood capillary walls in these two organs. Lung and brain microcapillaries consist of a contiguous endothelial cell layer with basement membrane whereas the capillaries in the liver and bone marrow, which are called sinusoids, consist of a fenestrated endothelial layer with discontinuous or absent basement membrane^{4,31}. The requirements for extravasation into the pulmonary and brain parenchymas therefore may be more stringent than for extravasation into the liver parenchyma or the bone marrow (FIG. 5D). The identification of a common set of genes in the BrMS and LMS, including genes that mediate extravasation, is consistent with this hypothesis, as is the finding that the BrMS and LMS are associated with relapse to brain and lung but not to bone or liver.

The similarities between brain and pulmonary metastasis notwithstanding, there are major differences in the structure of the capillary walls and the parenchyma of these two organs. The known functions encoded by the brain metastasis-associated genes identified here reflect those differences. Focusing on one of these genes, the brain sialyltransferase ST6GALNAC5, we find that its activity in breast cancer cells is required for BBB extravasation in vitro and brain metastasis in vivo. Cell-surface carbohydrates are regulators of cellular recognition processes and as such are thought to play important roles in the intercellular recognition events that occur during tumor progression²⁴ The present identification of ST6GALNAC5 as a gene expressed in breast cancer cells for BBB breaching draws attention to sialylated cell surface glycolipids as significant, previously unrecognized participants in brain metastasis.

The present findings open an opportunity for further delineation of the molecular and cellular mechanisms that underlie brain metastasis. We have focused here on the first step in this process, the passage of cancer cells through the BBB, and on some of the most salient mediators emerging from our functional and clinical screening. However, other genes identified in the present work are likely to play important roles in brain entry and colonization as well. IL-1 and TLR4 are known to induce BBB permeability and leukocyte extravasation in brain inflammatory processes³²⁻³⁵. The metalloprotease MMP3/stromelysin-1 mediates extracellular matrix degradation and growth factor mobilization, and has been implicated in brain metastasis in a rat syngeneic model³⁶⁻³⁷. Moreover, as in the case of ST6GALNAC5, some of these genes are primarily expressed in the brain: PRSS3/mesotrypsin is expressed in neurons and astrocytes and implicated in the activation of PAR-1 (protease-activated receptor-1)³⁸, Serpine-2 is secreted by glial cells and plays critical roles in synaptic plasticity 39 and differentiation of cerebellar granular neuron precursors⁴⁰. Neuroserpin is primarily secreted by axons in the brain and is thought to participate in synaptic plasticity and to have a neuroprotective role⁴¹. The functional relevance of these candidate mediators is now open to further analysis, as is the possibility that their blockade with specific inhibitors may stifle the seeding and outgrowth of brain metastases.

### Methods

Isolation of Carcinoma Cells from Pleural Effusions

Clinical specimens were obtained from three consenting patients (CN34, CN37, CN41) with metastatic breast cancer treated at our institution, following IRB-approved protocols. Epithelial cells were obtained from pleural fluids as described before⁴². Briefly, pleural fluid was collected in the presence of heparin (5 U/ml), and centrifuged at 1,000 rpm for 10 minutes. Cell pellets were resuspended in PBS, red blood cells were lyzed with ACK 1ysis buffer and a fraction of the cells was subjected to negative selection to remove leukocytes (CD45⁺ and CD15⁺ populations). Cells were cultured for 24 h to allow them to recover, and epithelial cells were sorted from this population using EpCam antibody. The resulting cell population was transduced with a lentivirus expressing the triple-fusion reporter encoding herpes simplex virus thymidine kinase 1, green fluorescent protein (GFP) and firefly luciferase⁴³. GFP-expressing cells were sorted and maintained at 5% CO₂ at 37° C. in M199 medium supplemented with 2.5% fetal bovine serum, 10 microg/ml insulin, 0.5 microg/ml hydrocortisone, 20 ng/ml EGF, 100 ng/ml cholera toxin, 1 microg/ml fungizone, and 100 U/ml penicillin/streptomycin, for approximately one week before mouse injection.

### Isolation of Brain Metastatic Cells

A cell suspension containing 10⁵ CN34 breast cancer cells in a volume of 100 microl was injected in the left cardiac ventricle of anesthetized 6-7 week old Cr:NIH-bg-nu-Xid mice. A cell suspension of 10⁴ MDA-MB-231 breast cancer cells in a volume of 100 microl was injected in the left cardiac ventricle of anesthetized 6-7 week old athymic mice. Tumor development was monitored by weekly bioluminescence imaging using the IVIS-200 imaging system from Xenogen as previously described⁹. Brain metastatic lesions were confirmed by magnetic resonance imaging (MRI), and histological analysis upon necropsy. Brain lesions were localized by ex vivo bioluminescence imaging, and resected under sterile conditions. Half of the tissue was fixed with 4% paraformaldehyde (PFA), and processed for histological analysis. The other half was minced and placed in culture medium containing a 1:1 mixture of Dulbecco's modified Eagle's (DME) medium/Ham's F12 supplemented with 0.125% collagenase III, 0.1% hyaluronidase. Samples were incubated at room temperature for 4-5 h, with gentle rocking. After collagenase treatment, cells were briefly centrifuged, resuspended in 0.25% trypsin, and incubated for an additional 15 min in a 37° C. water bath. Cells were resuspended in culture medium and allowed to grow to confluence on a 10 cm dish. GFP⁺ cells were sorted for further in vivo passage. All animal work was done following a protocol approved by the MSKCC Institutional Animal Care and Use Committee.

### Histological Analysis and Microscopy

Brain metastatic lesions were fixed with 4% PFA overnight, washed twice with PBS, dehydrated in ethanol 50%, and subsequent ethanol 70%, and embedded in paraffin for hematoxylin and eosin staining. For all other purposes, animals were perfused with 10 ml of PBS, and pre-fixed with 5 ml of 4% PFA. Lesions were extracted and post-fixed with 4% PFA for 2 additional hours, incubated in a solution of 30% sucrose in PBS for 1-2 days, and processed for OCT compound embedding and montage. Assessment of reactive glia was performed by staining with the astrocyte marker glial fibrillary protein (GFAP, DAKO), followed by detection with fluorescently labeled secondary antibody. Microscopic analysis was performed using a Zeiss Axioplan2 microscope.

For detection of tumor cells in the brain microvasculature, 10⁶ brain metastatic cells were injected into the left ventricle of anesthetized mice. Enhancement of the green fluorescence was obtained by labeling the tumor cells with 5 microM CFMDA cell tracker dye (Invitrogen) for 45 min before injection. Mice were injected with 2 mg/g of body weight rodhamin-labeled 70 kDa dextran (Invitrogen) via retro-orbital inoculation one hour before sacrifice to stain the brain vasculature. Animals were perfused and sacrificed 24 h after tumor cell inoculation, and brain was processed for OCT compound embedding. 30 microm sections were examined using an Upright Leica TCS SP2 confocal microscope, and 63× images were collected beta

### RNA Isolation and Gene-Expression Profiling

RNA was extracted from exponentially growing cells using the RNeasy mini kit (Qiagen). Labeling and hybridization of the samples to HG-U133A gene expression chip (Affymetrix) was performed by the MSKCC Genomics Core Facility using standard methodology. Data analysis was performed using the GeneSpring 7.2 software. The raw data was filtered by intensity values equal or larger than 150. Class comparison between parental and brain metastatic populations was performed, and the gene list was filtered by Student's t-test of the 2.5 fold differentially expressed genes.

RNA extraction, labeling and hybridization of clinical samples for microarray analysis was done as previously described¹².

### BrMS Derivation and Clinical Sample Analysis

Microarray data from four cohorts of breast tumors were used for analysis. The MSK-82 cohort was more locally advanced compared to either the NKI-295 or the EMC-286 series (91% T2-T4 and 66% node positive in MSK-82, compared to 47% T2-T4 and 49% node positive in NKI-295, and 51% T1, 46% T2 and 0% node positive in EMC-286). EMC-204 is a heterogeneous cohort that includes 156 tumors from patients that relapsed and received first-line chemotherapy for metastatic disease and 48 from patients that were node-negative and did not receive adjuvant systemic therapy.

The MSK-82 and EMC-286 cohorts were analyzed on Affymetrix HG-U133A platform, and the EMC-204 cohort on HG-U133 plus 2.0. The NKI-295 set was analyzed on Agilent microarrays. To achieve statistical power given the limited incidence of brain metastasis in these cohorts, we merged MSK-82 and EMC-286 cohorts. All datasets were first transformed to log 2 scales and median-centered. Z-transformation was then performed to normalize gene expression across all samples in each cohort⁴⁴.

The gene-expression associated with brain metastasis in each model system was used to fit a Cox hazard ratio regression model to gauge the association of each gene with brain- or lung-metastasis free survival in the MSK-82/EMC-286 cohort. This was achieved using the survival package in the R statistical software. Wald test was used to calculate the p-values. We designated Brain Metastasis gene-expression Signature (BrMS) the 17 genes with p-values <0.05 that fulfill any of the following criteria: genes that are selected in the same direction in the CN34 and MDA231 BrM cells systems by >1.5 fold; genes that are upregulated in one system and maintain high levels in the other; genes that are downregulated in one system and maintain at low levels in the other. The identification of BrMS+ tumors was achieved by unsupervised hierarchical clustering of tumors in the MSK-82/EMC-286 as a training cohort. The resulting cluster-tree was cut at different distance cutoffs to yield different numbers (2 to 10) of sub-clusters. In each case, the cluster that most resembled the gene expression pattern of BrM cells was compared with the other clusters for enrichment of brain relapse events, using Fisher's exact test. The best cutoff was determined when such cluster not only maintained the resemblance of gene expression pattern to BrM cells, but also best segregated brain relapse events. This cluster was defined as BrMS+. Heatmaps were generated using the gplots package of R statistical program.

BrMS+ and BrMS− (i.e., non-BrMS+) tumors were used to train a support vector machine (package e1071, R statistical program). We employed a linear kernel and used expression values of the 17-gene BrMS as features. The trained classifier was then applied to the NKI-295 and EMC-204 cohorts to predict BrMS+ tumors. We performed Kaplan-Meier analysis and log-rank test on the survival rates of the predicted BrMS+ and BrMS− tumors in the NKI-295 and EMC-204 datasets, using the survival package of R.

### Knockdown Cell Lines

Knockdown of COX-2 and EREG with a validated hairpin was achieved as previously described¹⁴.Knockdown of HB-EGF was achieved with pRetroSuper vector targeting the sequence 5′-GGTATGCTGTCATGGTCCT-3′ (Seq. ID No. 3), and knockdown of ST6GALNAC5 by targeting the sequences 5′-CATAAGCAACTCAACAATA-3′ (Seq. ID No. 1) (shRNA2), and 5′-AGCACATCTCCACTGACT-3′ (Seq ID No. 2) (shRNA3). The efficiency of the knock down was confirmed by qRT-PCR TaqMan gene expression assays (Applied Biosystems), or western immunoblotting analysis (Cox-2, Cayman antibody). Beta-2 microglobulin and actin were used as endogenous controls for qRT-PCR and western blot, respectively. The viral particles for infection of the brain metastatic derivatives were obtained by transfection of the GPG29 amphotropic packaging cell line, and collection supernatants at 48 and 72 h after transfection. Supernatants were filtered and centrifuged at 19,000 rpm to concentrate the viral particles, and used to infect sub confluent cultures in the presence of 5 microg/ml polybrene overnight. Puromycin (2 microg/ml) was used to select for stable cell lines. Only cell lines with a transduction rate over 80-90% were used for further studies.

### Cetuximab Treatment

Biweekly intraperitoneal injection of 1 mg of cetuximab antibody (ImClone) was performed as previously described¹⁴. Animals were given one or two doses of cetuximab before intracardiac inoculation of the tumor cells, and were maintained on drug treatment until the end of the experiment.

### In Vitro Blood-Brain Barrier Assay

Primary human umbilical vein endothelial cells (ScienCell) were co-cultured with human primary astrocytes (HA, ScienCell), on opposite sides of a polylysin-treated, gelatin-coated tissue culture trans-well insert for three days as previously described²⁰. Briefly, 3 microm pore PET tissue culture inserts (Fisher) were treated with polylysine (1 microg/ml, Millipore) overnight, washed four times, and coated with 0.2% gelatin (Sigma) for a minimum of 30 min. Inserts were placed upside-down in a 15 cm plate, and 10⁵ primary human astrocytes were plated on the membrane surface. Astrocytes were fed every 15 minutes for 5 h, and inserts were then flipped and placed in 24-well plates. 5×10⁴ endothelial cells were plated on the upper chamber of the inserts, and cultures were placed in the incubator, without further perturbation. Three days later, the tightness of the barriers was tested by permeability to serum albumin. Evans blue-conjugated albumin (0.45% in phenol red medium) was added to the upper chamber and incubated for 30 min at 37° C. Medium from the bottom chamber was collected, and absorbance was measured at 620 nm. Controls include astrocytes alone, endothelial cells alone, astrocytes plated on both sides of the insert, and insert alone. Specific staining of each monolayer was done by using the endothelial cell markers von Willebrand factor (vWF, Sigma) and the astrocyte marker GFAP (Dako).

For BBB transmigration assays, cancer cells were labeled with 5 microM CFMDA cell tracker green (Invitrogen) for 45 min, and recovered overnight before assaying. 5×10⁴ cells were seeded on the upper chamber and incubated for 14-18 h. Inserts were washed with PBS, fixed with 4% PFA for 20 min; subsequently the membranes were removed from the plastic insert and mounted on a microscope slide. 5× pictures from 5-8 inserts per experiment were taken, and the number of transmigrated cells was counted.

### Cell Adhesion Assay

Primary human brain microvascular endothelial cells (hBMVECs, ScienCell) were grown to confluency in 12-well plates. Before seeding the tumor cells, hBMVEC monolayers were washed twice with 0.5% bovine serum albumin (BSA) PBS. Tumor cells were briefly trypsinized, resuspended in medium containing 0.5% BSA, and counted. 5×10⁵ cells were plated in each well, and allowed to adhere to the monolayer for 30 minutes. Plates were washed 3 times for 5 min each, shaking. Cells were lysed with 100 microl with 1× Passive lysis buffer (Promega) for 1 h, shaking. Firefly luciferase activity was determined using an Orion microplate luminometer (Berthold Detection Systems). Assays were performed in quadruplicates.

### Lectin Staining

Sambucus nigra agglutinin (SNA) staining was performed on perfused, paraffin embedded xenograft tumor tissue. Briefly, after standard deparafinization, sections were washed with PBS, and endogenous peroxidase was quenched by incubation in 0.3% H₂O₂ in methanol for 30 minutes at room temperature. Sections were washed three times with PBS and blocked in 10% donkey serum for 30 min at room temperature. Labeling with biotin-conjugated SNA was carried out at a concentration of 100 microg/ml for 45 min, followed by 3 washes with PBS. An Alexa-568 conjugated-tyramide amplification kit (Invitrogen) was used following manufacturer's procedures to detect the biotinilated lectin. Sections were mounted with Prolong Gold mounting medium (Invitrogen), and images were taken using a Zeiss Axioplan2 microscope. The same protocol was followed for SNA staining of human breast cancer metastatic tissues, except that SNA was used at 10 microg/ml.

### Other Tissue Culture Procedures.

Primary human endothelial cells and astrocytes were cultured in M199 medium supplemented with 50 mg/ml ascorbic acid, 25 mg/ml heparin, 3 mg/ml endothelial cell growth supplement (Sigma), 5 microg/ml bovine brain extract (Clonetics), 20% fetal bovine serum (FBS), 5% human serum (Biocell), 1 microg/ml fungizone, and 100 U/ml penicillin/streptomycin. GPG29 cells were cultured in DME supplemented with 20 ng/ml doxycycline, 2 microg/ml puromycin, 0.3 mg/ml G418, and 10% FBS. 293T/17 packaging cell lines used for lentiviral production, and MDA-MB-231 parental cell lines and derivatives were cultured in DME supplemented with 10% FBS, 1 microg/ml fungizone, and 100 U/ml penicillin/streptomycin. All transfections were performed using Lipofectamine2000 (Invitrogen). GPG29 cells were maintained in DME supplemented with 10% FBS and 1 mM sodium pyruvate after transfection.

### Oncomine Gene Expression Data Analysis

Relative levels of ST6GalNac5 mRNA expression in human tissues was obtained by Oncomine Cancer Microarray database analysis (http://www.oncomine.org)⁴⁵ of a published gene expression dataset⁴⁶. The data was log 2-transformed, with the media set to zero and standard deviation set to one. p-values were calculated based on Student's t-test.

TABLE 1
Brain metastasis gene-expression Signature (BrMS). Group of 17 genes with
significant association with brain metastasis-free survival in the MSK-82/EMC-286
(training set), EMC-204, and NKI-295 (independent datasets). p-values of
univariate correlation with brain metastasis-free survival in the combined
MSK-82/EMC-286 dataset are shown for each of the genes.

| | | | |
|---|---|---|---|
| | Gene | | |
| Probe | Symbol | Gene Name | p |

Downregulated genes

| | | | |
|---|---|---|---|
| 202688_at | TNFSF10 | tumor necrosis factor superfamily, member 10; TRAIL | 0.0006 |
| 204070_at | RARRES3 | Retinoic acid receptor responder (tazarotene induced) 3 | 0.00105 |
| 203453_at | SCNN1A | sodium channel, nonvoltage-gated 1 alpha | 0.00629 |
| 201427_s_at | SEPP1 | selenoprotein P, plasma, 1 | 0.04811 |

Upregulated genes

| | | | |
|---|---|---|---|
| 221009_s_at | ANGPTL4 | angiopoietin-like 4 | 0.00129 |
| 202620_s_at | PLOD2 | procollagen-lysine, 2-oxoglutarate 5-dioxygenase 2 | 0.00322 |
| 211343_s_at | COL13A1 | collagen, type XIII, alpha 1 | 0.00378 |
| 204748_at | PTGS2 | prostaglandin-endoperoxide synthase 2 (prostaglandin | 0.01054 |
| | | G/H synthase and cyclooxygenase); COX-2 | |
| 218319_at | PELI1 | pellino homolog 1 (Drosophila) | 0.02117 |
| 204475_at | MMP1 | matrix metallopeptidase 1 (interstitial collagenase) | 0.02631 |
| 206233_at | B4GALT6 | UDP-Gal:betaGlcNAc beta 1,4-galactosyltransferase, | 0.02927 |
| | | polypeptide 6 | |
| 203821_at | HBEGF | heparin-binding EGF-like growth factor | 0.0294 |
| 207442_at | CSF3 | colony stimulating factor 3 (granulocyte) | 0.02985 |
| 218723_s_at | RGC32 | response to complement 32 | 0.03569 |
| 202728_s_at | LTBP1 | latent transforming growth factor beta binding protein 1 | 0.04207 |
| 201564_s_at | FSCN1 | fascin homolog 1, actin-bundling protein | 0.04399 |
| | | (Strongylocentrotus purpuratus) | |
| 202202_s_at | LAMA4 | laminin, alpha 4 | 0.04986 |

TABLE 2
Top scoring genes from a class comparison between parental and highly brain
metastatic cells in CN34 and MDA231 systems. List of 18 genes (corresponding to 19 probe
sets) obtained with a filter of 3-fold increase, after exclusion of histones and previously
identified bone metastasis and lung metastasis-associated genes. The fold-change in gene
expression in CN34 or MDA231 BrM cells versus parental cells is indicated.

| Probe | Gene Symbol | Gene Name | CN34 | MDA231 |
|---|---|---|---|---|
| 205534_at | PCDH7 | protocadherin 7 | 50.06 | 7.264 |
| 201288_at | ARHGDIB | Rho GDP dissociation inhibitor (GDI) beta | 7.658 | 4.185 |
| 220979_s_at | ST6GALNAC5 | ST6 (alpha-N-acetyl-neuraminyl-2,3-beta- | 7.456 | 3.028 |
| | | galactosyl-1,3)-N-acetylgalactosaminide | | |
| | | alpha-2,6-sialyltransferase 5 | | |
| 219523_s_at | ODZ3 | odz, odd Oz/ten-m homolog 3 (Drosophila) | 7.333 | 3.312 |
| 205352_at | SERPINI1 | serine (or cysteine) proteinase inhibitor, | 7.1 | 3.539 |
| | | clade I (neuroserpin), member 1 | | |
| 201667_at | GJA1 | gap junction protein, alpha 1, 43 kDa | 5.784 | 3.291 |
| | | (connexin 43) | | |
| 213280_at | GARNL4 | GTPase activating RANGAP domain-like 4 | 5.607 | 10.97 |
| 207463_x_at | PRSS3 | protease, serine, 3 (mesotrypsin) | 5.247 | 4.028 |
| 212190_at | SERPINE2 | serpin peptidase inhibitor, clade E (nexin, | 5.149 | 3.238 |
| | | plasminogen activator inhibitor type 1), | | |
| | | member 2 | | |
| 218587_s_at | KTELC1 | KTEL (Lys-Tyr-Glu-Leu) containing 1 | 4.734 | 3.249 |
| 202388_at | RGS2 | regulator of G-protein signalling 2, 24 kDa | 4.566 | 4.887 |
| 205067_at | IL1B | interleukin 1, beta | 4.396 | 9.563 |
| 39402_at | IL1B | interleukin 1, beta | 4.324 | 4.671 |
| 205828_at | MMP3 | matrix metalloproteinase 3 (stromelysin 1, | 4.305 | 5.214 |
| | | progelatinase) | | |
| 220169_at | TMEM156 | transmembrane protein 156 | 4.127 | 3.009 |
| 219377_at | FAM59A | family with sequence similarity 59, member A | 4.081 | 3.639 |
| 210118_s_at | IL1A | interleukin 1, alpha | 3.409 | 3.551 |
| 213181_s_at | MOCS1 | molybdenum cofactor synthesis 1 | 3.357 | 3.713 |
| 221060_s_at | TLR4 | toll-like receptor 4 | 3.345 | 3.033 |

TABLE 3
Class comparison between parental CN34 and brain metastatic
derivatives. These 310 probe sets represent 271 genes
differentially expressed by more than 2.5 fold between the
two classes. Fold change values are indicated.

| | Fold | | |
|---|---|---|---|
| Probe set | Change | Gene symbol | Gene title |
| 209173_at | 116.2 | AGR2 | anterior gradient homolog 2 (Xenopus laevis) |
| 204475_at | 102.1 | MMP1 | matrix metallopeptidase 1 (interstitial |
| | | | collagenase) |
| 213194_at | 53.43 | ROBO1 | roundabout, axon guidance receptor, homolog 1 |
| | | | (Drosophila) |
| 205534_at | 50.06 | PCDH7 | protocadherin 7 |
| 206224_at | 47.11 | CST1 | cystatin SN |
| 221760_at | 27.83 | MAN1A1 | Mannosidase, alpha, class 1A, member 1 |
| 204748_at | 21.1 | PTGS2 | prostaglandin-endoperoxide synthase 2 |
| | | | (prostaglandin G/H synthase and |
| | | | cyclooxygenase) |
| 204749_at | 19.84 | NAP1L3 | nucleosome assembly protein 1-like 3 |
| 205969_at | 18.84 | AADAC | arylacetamide deacetylase (esterase) |
| 203895_at | 17.08 | PLCB4 | phospholipase C, beta 4 |
| 206218_at | 15.41 | MAGEB2 | melanoma antigen family B, 2 |
| 218723_s_at | 14.67 | C13orf15 | chromosome 13 open reading frame 15 |
| 204078_at | 13.06 | SC65 | synaptonemal complex protein SC65 |
| 203896_s_at | 12.89 | PLCB4 | phospholipase C, beta 4 |
| 214455_at | 12.6 | HIST1H2BC | histone cluster 1, H2bc |
| 202947_s_at | 12.49 | GYPC | glycophorin C (Gerbich blood group) |
| 202158_s_at | 11.83 | CUGBP2 | CUG triplet repeat, RNA binding protein 2 |
| 205945_at | 11.63 | IL6R | interleukin 6 receptor |
| 221558_s_at | 10.34 | LEF1 | lymphoid enhancer-binding factor 1 |
| 218338_at | 10.16 | LOC653441 | polyhomeotic homolog 1 (Drosophila) |
| 205259_at | 10.1 | NR3C2 | nuclear receptor subfamily 3, group C, member 2 |
| 205289_at | 9.916 | BMP2 | bone morphogenetic protein 2 |
| 205476_at | 9.747 | CCL20 | chemokine (C-C motif) ligand 20 |
| 203122_at | 9.423 | TTC15 | tetratricopeptide repeat domain 15 |
| 214963_at | 9.318 | NUP160 | nucleoporin 160 kDa |
| 220088_at | 8.219 | C5AR1 | complement component 5a receptor 1 |
| 214404_x_at | 8.058 | SPDEF | SAM pointed domain containing ets transcription |
| | | | factor |
| 211367_s_at | 7.869 | CASP1 | caspase 1, apoptosis-related cysteine peptidase |
| | | | (interleukin 1, beta, convertase) |
| 211368_s_at | 7.868 | CASP1 | caspase 1, apoptosis-related cysteine peptidase |
| | | | (interleukin 1, beta, convertase) |
| 213618_at | 7.781 | CENTD1 | centaurin, delta 1 |
| 201288_at | 7.658 | ARHGDIB | Rho GDP dissociation inhibitor (GDI) beta |
| 220979_s_at | 7.456 | ST6GALNAC5 | ST6 (alpha-N-acetyl-neuraminyl-2,3-beta- |
| | | | galactosyl-1,3)-N-acetylgalactosaminide alpha- |
| | | | 2,6-sialyltransferase 5 |
| 205576_at | 7.399 | SERPIND1 | serpin peptidase inhibitor, clade D (heparin |
| | | | cofactor), member 1 |
| 219667_s_at | 7.372 | BANK1 | B-cell scaffold protein with ankyrin repeats 1 |
| 219523_s_at | 7.333 | ODZ3 | odz, odd Oz/ten-m homolog 3 (Drosophila) |
| 205602_x_at | 7.305 | PSG7 | pregnancy specific beta-1-glycoprotein 7 |
| 207797_s_at | 7.156 | LRP2BP | LRP2 binding protein |
| 205352_at | 7.1 | SERPINI1 | serpin peptidase inhibitor, clade I (neuroserpin), |
| | | | member 1 |
| 201417_at | 6.567 | SOX4 | SRY (sex determining region Y)-box 4 |
| 201564_s_at | 6.363 | FSCN1 | fascin homolog 1, actin-bundling protein |
| | | | (Strongylocentrotus purpuratus) |
| 205302_at | 6.32 | IGFBP1 | insulin-like growth factor binding protein 1 |
| 221009_s_at | 6.104 | ANGPTL4 | angiopoietin-like 4 |
| 206295_at | 6.081 | IL18 | interleukin 18 (interferon-gamma-inducing |
| | | | factor) |
| 208322_s_at | 5.961 | ST3GAL1 | ST3 beta-galactoside alpha-2,3-sialyltransferase 1 |
| 204567_s_at | 5.946 | ABCG1 | ATP-binding cassette, sub-family G (WHITE), |
| | | | member 1 |
| 208609_s_at | 5.904 | TNXA | tenascin XA pseudogene |
| 209755_at | 5.803 | NMNAT2 | nicotinamide nucleotide adenylyltransferase 2 |
| 201667_at | 5.784 | GJA1 | gap junction protein, alpha 1, 43 kDa |
| 204454_at | 5.684 | LDOC1 | leucine zipper, down-regulated in cancer 1 |
| 213280_at | 5.607 | GARNL4 | GTPase activating Rap/RanGAP domain-like 4 |
| 206191_at | 5.536 | ENTPD3 | ectonucleoside triphosphate diphosphohydrolase 3 |
| 215071_s_at | 5.517 | HIST1H2AC | histone cluster 1, H2ac |
| 209360_s_at | 5.396 | RUNX1 | runt-related transcription factor 1 (acute myeloid |
| | | | leukemia 1; aml1 oncogene) |
| 205402_x_at | 5.389 | PRSS2 | protease, serine, 2 (trypsin 2) |
| 207463_x_at | 5.247 | PRSS3 | protease, serine, 3 (mesotrypsin) |
| 212190_at | 5.149 | SERPINE2 | serpin peptidase inhibitor, clade E (nexin, |
| | | | plasminogen activator inhibitor type 1), member 2 |
| 218280_x_at | 4.937 | HIST2H2AA3 | histone cluster 2, H2aa3 |
| 214290_s_at | 4.855 | HIST2H2AA3 | histone cluster 2, H2aa3 |
| 208978_at | 4.836 | CRIP2 | cysteine-rich protein 2 |
| 206609_at | 4.776 | MAGEC1 | melanoma antigen family C, 1 |
| 205342_s_at | 4.763 | SULT1C2 | sulfotransferase family, cytosolic, 1C, member 2 |
| 218587_s_at | 4.734 | KTELC1 | KTEL (Lys-Tyr-Glu-Leu) containing 1 |
| 212819_at | 4.712 | ASB1 | ankyrin repeat and SOCS box-containing 1 |
| 211366_x_at | 4.633 | CASP1 | caspase 1, apoptosis-related cysteine peptidase |
| | | | (interleukin 1, beta, convertase) |
| 212154_at | 4.602 | SDC2 | syndecan 2 |
| 202388_at | 4.566 | RGS2 | regulator of G-protein signaling 2, 24 kDa |
| 209114_at | 4.526 | TSPAN1 | tetraspanin 1 |
| 207968_s_at | 4.506 | MEF2C | myocyte enhancer factor 2C |
| 204339_s_at | 4.437 | RGS4 | regulator of G-protein signaling 4 |
| 205067_at | 4.396 | IL1B | interleukin 1, beta |
| 218752_at | 4.391 | ZMAT5 | zinc finger, matrin type 5 |
| 209970_x_at | 4.389 | CASP1 | caspase 1, apoptosis-related cysteine peptidase |
| | | | (interleukin 1, beta, convertase) |
| 213421_x_at | 4.354 | PRSS3 | protease, serine, 3 (mesotrypsin) |
| 219047_s_at | 4.337 | ZNF668 | zinc finger protein 668 |
| 39402_at | 4.324 | IL1B | interleukin 1, beta |
| 205828_at | 4.305 | MMP3 | matrix metallopeptidase 3 (stromelysin 1, |
| | | | progelatinase) |
| 207199_at | 4.297 | TERT | telomerase reverse transcriptase |
| 206924_at | 4.219 | IL11 | interleukin 11 |
| 219132_at | 4.209 | PELI2 | pellino homolog 2 (Drosophila) |
| 220945_x_at | 4.166 | MANSC1 | MANSC domain containing 1 |
| 204623_at | 4.152 | TFF3 | trefoil factor 3 (intestinal) |
| 220169_at | 4.127 | TMEM156 | transmembrane protein 156 |
| 215395_x_at | 4.096 | TRY6 | trypsinogen C |
| 217087_at | 4.081 | C1orf68 | chromosome 1 open reading frame 68 |
| 219377_at | 4.081 | FAM59A | family with sequence similarity 59, member A |
| 219032_x_at | 4.031 | OPN3 | opsin 3 (encephalopsin, panopsin) |
| 201397_at | 4.028 | PHGDH | phosphoglycerate dehydrogenase |
| 210102_at | 4.02 | LOH11CR2A | loss of heterozygosity, 11, chromosomal region |
| | | | 2, gene A |
| 209561_at | 4.011 | THBS3 | thrombospondin 3 |
| 210993_s_at | 3.994 | SMAD1 | SMAD family member 1 |
| 219322_s_at | 3.966 | WDR8 | WD repeat domain 8 |
| 201998_at | 3.963 | ST6GAL1 | ST6 beta-galactosamide alpha-2,6- |
| | | | sialyltranferase 1 |
| 207379_at | 3.963 | EDIL3 | EGF-like repeats and discoidin I-like domains 3 |
| 202157_s_at | 3.959 | CUGBP2 | CUG triplet repeat, RNA binding protein 2 |
| 201008_s_at | 3.933 | TXNIP | thioredoxin interacting protein |
| 206011_at | 3.926 | CASP1 | caspase 1, apoptosis-related cysteine peptidase |
| | | | (interleukin 1, beta, convertase) |
| 208378_x_at | 3.917 | FGF5 | fibroblast growth factor 5 |
| 209199_s_at | 3.903 | MEF2C | myocyte enhancer factor 2C |
| 205767_at | 3.877 | EREG | epiregulin |
| 213441_x_at | 3.877 | SPDEF | SAM pointed domain containing ets transcription |
| | | | factor |
| 204977_at | 3.862 | DDX10 | DEAD (Asp-Glu-Ala-Asp) box polypeptide 10 |
| 222105_s_at | 3.797 | NKIRAS2 | NFKB inhibitor interacting Ras-like 2 |
| 203821_at | 3.746 | HBEGF | heparin-binding EGF-like growth factor |
| 210933_s_at | 3.735 | FSCN1 | fascin homolog 1, actin-bundling protein |
| | | | (Strongylocentrotus purpuratus) |
| 212448_at | 3.704 | NEDD4L | neural precursor cell expressed, developmentally |
| | | | down-regulated 4-like |
| 209781_s_at | 3.645 | KHDRBS3 | KH domain containing, RNA binding, signal |
| | | | transduction associated 3 |
| 216235_s_at | 3.606 | EDNRA | endothelin receptor type A |
| 222067_x_at | 3.581 | HIST1H2BD | histone cluster 1, H2bd |
| 214696_at | 3.571 | C17orf91 | chromosome 17 open reading frame 91 |
| 202202_s_at | 3.568 | LAMA4 | laminin, alpha 4 |
| 204679_at | 3.548 | KCNK1 | potassium channel, subfamily K, member 1 |
| 209911_x_at | 3.515 | HIST1H2BD | histone cluster 1, H2bd |
| 221044_s_at | 3.478 | TRIM34 | tripartite motif-containing 34 |
| 212158_at | 3.435 | SDC2 | syndecan 2 |
| 219156_at | 3.418 | SYNJ2BP | synaptojanin 2 binding protein |
| 210118_s_at | 3.409 | IL1A | interleukin 1, alpha |
| 213181_s_at | 3.357 | MOCS1 | molybdenum cofactor synthesis 1 |
| 221060_s_at | 3.345 | TLR4 | toll-like receptor 4 |
| 206233_at | 3.344 | B4GALT6 | UDP-Gal: betaGlcNAc beta 1,4- |
| | | | galactosyltransferase, polypeptide 6 |
| 213489_at | 3.341 | MAPRE2 | Microtubule-associated protein, RP/EB family, |
| | | | member 2 |
| 203372_s_at | 3.332 | SOCS2 | suppressor of cytokine signaling 2 |
| 48825_at | 3.323 | ING4 | inhibitor of growth family, member 4 |
| 205290_s_at | 3.317 | BMP2 | bone morphogenetic protein 2 |
| 209398_at | 3.311 | HIST1H1C | histone cluster 1, H1c |
| 202796_at | 3.292 | SYNPO | synaptopodin |
| 211343_s_at | 3.28 | COL13A1 | collagen, type XIII, alpha 1 |
| 202708_s_at | 3.276 | HIST2H2BE | histone cluster 2, H2be |
| 205224_at | 3.255 | SURF2 | surfeit 2 |
| 209099_x_at | 3.246 | JAG1 | jagged 1 (Alagille syndrome) |
| 210396_s_at | 3.218 | BOLA2 | PI-3-kinase-related kinase SMG-1 pseudogene |
| 216268_s_at | 3.213 | JAG1 | jagged 1 (Alagille syndrome) |
| 203180_at | 3.189 | ALDH1A3 | aldehyde dehydrogenase 1 family, member A3 |
| 211653_x_at | 3.175 | AKR1C2 | aldo-keto reductase family 1, member C2 |
| | | | (dihydrodiol dehydrogenase 2; bile acid binding |
| | | | protein; 3-alpha hydroxysteroid dehydrogenase, |
| | | | type III) |
| 219911_s_at | 3.17 | SLCO4A1 | solute carrier organic anion transporter family, |
| | | | member 4A1 |
| 208579_x_at | 3.136 | H2BFS | H2B histone family, member S |
| 218688_at | 3.107 | DAK | dihydroxyacetone kinase 2 homolog (S. cerevisiae) |
| 38037_at | 3.102 | HBEGF | heparin-binding EGF-like growth factor |
| 218362_s_at | 3.082 | DIS3 | DIS3 mitotic control homolog (S. cerevisiae) |
| 206953_s_at | 3.079 | LPHN2 | latrophilin 2 |
| 220192_x_at | 3.055 | SPDEF | SAM pointed domain containing ets transcription |
| | | | factor |
| 204523_at | 3.046 | ZNF140 | zinc finger protein 140 |
| 206051_at | 3.043 | ELAVL4 | ELAV (embryonic lethal, abnormal vision, |
| | | | Drosophila)-like 4 (Hu antigen D) |
| 208595_s_at | 3.031 | MBD1 | methyl-CpG binding domain protein 1 |
| 214434_at | 3.029 | HSPA12A | heat shock 70 kDa protein 12A |
| 208146_s_at | 3.011 | CPVL | carboxypeptidase, vitellogenic-like |
| 204161_s_at | 3.003 | ENPP4 | ectonucleotide |
| | | | pyrophosphatase/phosphodiesterase 4 (putative |
| | | | function) |
| 219916_s_at | 2.999 | RNF39 | ring finger protein 39 |
| 205266_at | 2.983 | LIF | leukemia inhibitory factor (cholinergic |
| | | | differentiation factor) |
| 212936_at | 2.97 | C5orf21 | chromosome 5 open reading frame 21 |
| 201280_s_at | 2.959 | DAB2 | disabled homolog 2, mitogen-responsive |
| | | | phosphoprotein (Drosophila) |
| 205331_s_at | 2.949 | REEP2 | receptor accessory protein 2 |
| 218952_at | 2.941 | PCSK1N | proprotein convertase subtilisin/kexin type 1 |
| | | | inhibitor |
| 209310_s_at | 2.94 | CASP4 | caspase 4, apoptosis-related cysteine peptidase |
| 221650_s_at | 2.94 | MED18 | mediator complex subunit 18 |
| 204151_x_at | 2.929 | AKR1C1 | aldo-keto reductase family 1, member C1 |
| | | | (dihydrodiol dehydrogenase 1; 20-alpha (3- |
| | | | alpha)-hydroxysteroid dehydrogenase) |
| 209098_s_at | 2.912 | JAG1 | jagged 1 (Alagille syndrome) |
| 205136_s_at | 2.893 | NUFIP1 | nuclear fragile X mental retardation protein |
| | | | interacting protein 1 |
| 212992_at | 2.881 | AHNAK2 | AHNAK nucleoprotein 2 |
| 210336_x_at | 2.879 | MZF1 | myeloid zinc finger 1 |
| 204417_at | 2.872 | GALC | galactosylceramidase |
| 202438_x_at | 2.848 | IDS | iduronate 2-sulfatase (Hunter syndrome) |
| 221530_s_at | 2.819 | BHLHB3 | basic helix-loop-helix domain containing, class |
| | | | B, 3 |
| 207542_s_at | 2.812 | AQP1 | aquaporin 1 (Colton blood group) |
| 210736_x_at | 2.81 | DTNA | dystrobrevin, alpha |
| 203373_at | 2.807 | SOCS2 | suppressor of cytokine signaling 2 |
| 206342_x_at | 2.801 | IDS | iduronate 2-sulfatase (Hunter syndrome) |
| 209765_at | 2.8 | ADAM19 | ADAM metallopeptidase domain 19 (meltrin |
| | | | beta) |
| 211855_s_at | 2.796 | SLC25A14 | solute carrier family 25 (mitochondrial carrier, |
| | | | brain), member 14 |
| 219617_at | 2.792 | C2orf34 | chromosome 2 open reading frame 34 |
| 217631_at | 2.779 | GTPBP4 | GTP binding protein 4 |
| 217466_x_at | 2.777 | LOC400963 | ribosomal protein S2 |
| 221797_at | 2.777 | C17orf90 | chromosome 17 open reading frame 90 |
| 207601_at | 2.771 | SULT1B1 | sulfotransferase family, cytosolic, 1B, member 1 |
| 201010_s_at | 2.764 | TXNIP | thioredoxin interacting protein |
| 215498_s_at | 2.746 | MAP2K3 | mitogen-activated protein kinase kinase 3 |
| 218218_at | 2.744 | APPL2 | adaptor protein, phosphotyrosine interaction, PH |
| | | | domain and leucine zipper containing 2 |
| 219024_at | 2.728 | PLEKHA1 | pleckstrin homology domain containing, family |
| | | | A (phosphoinositide binding specific) member 1 |
| 208798_x_at | 2.71 | GOLGA8A | golgi autoantigen, golgin subfamily a, 8A |
| 206390_x_at | 2.705 | PF4 | platelet factor 4 (chemokine (C—X—C motif) |
| | | | ligand 4) |
| 209486_at | 2.673 | UTP3 | UTP3, small subunit (SSU) processome |
| | | | component, homolog (S. cerevisiae) |
| 203887_s_at | 2.651 | THBD | thrombomodulin |
| 218647_s_at | 2.644 | YRDC | yrdC domain containing (E. coli) |
| 50374_at | 2.635 | C17orf90 | chromosome 17 open reading frame 90 |
| 219848_s_at | 2.621 | ZNF432 | zinc finger protein 432 |
| 218307_at | 2.585 | RSAD1 | radical S-adenosyl methionine domain |
| | | | containing 1 |
| 212221_x_at | 2.562 | IDS | iduronate 2-sulfatase (Hunter syndrome) |
| 212766_s_at | 2.535 | ISG20L2 | interferon stimulated exonuclease gene 20 kDa- |
| | | | like 2 |
| 205019_s_at | 2.502 | VIPR1 | vasoactive intestinal peptide receptor 1 |
| 201904_s_at | 0.394 | CTDSPL | CTD (carboxy-terminal domain, RNA |
| | | | polymerase II, polypeptide A) small |
| | | | phosphatase-like |
| 214701_s_at | 0.392 | FN1 | fibronectin 1 |
| 205240_at | 0.391 | GPSM2 | G-protein signaling modulator 2 (AGS3-like, C. elegans) |
| 201093_x_at | 0.387 | SDHA | succinate dehydrogenase complex, subunit A, |
| | | | flavoprotein (Fp) |
| 201106_at | 0.387 | GPX4 | glutathione peroxidase 4 (phospholipid |
| | | | hydroperoxidase) |
| 204765_at | 0.386 | ARHGEF5 | Rho guanine nucleotide exchange factor (GEF) 5 |
| 201109_s_at | 0.385 | THBS1 | thrombospondin 1 |
| 219491_at | 0.381 | LRFN4 | leucine rich repeat and fibronectin type III |
| | | | domain containing 4 |
| 204391_x_at | 0.38 | TRIM24 | tripartite motif-containing 24 |
| 210916_s_at | 0.38 | CD44 | CD44 molecule (Indian blood group) |
| 204537_s_at | 0.379 | GABRE | gamma-aminobutyric acid (GABA) A receptor, |
| | | | epsilon |
| 218200_s_at | 0.379 | NDUFB2 | NADH dehydrogenase (ubiquinone) 1 beta |
| | | | subcomplex, 2, 8 kDa |
| 201028_s_at | 0.372 | CD99 | CD99 molecule |
| 200950_at | 0.369 | ARPC1A | actin related protein 2/3 complex, subunit 1A, |
| | | | 41 kDa |
| 205588_s_at | 0.369 | FGFR1OP | FGFR1 oncogene partner |
| 201329_s_at | 0.366 | ETS2 | v-ets erythroblastosis virus E26 oncogene |
| | | | homolog 2 (avian) |
| 217436_x_at | 0.366 | LOC730399 | hypothetical protein LOC730399 |
| 218303_x_at | 0.364 | KRCC1 | lysine-rich coiled-coil 1 |
| 202642_s_at | 0.362 | TRRAP | transformation/transcription domain-associated |
| | | | protein |
| 209587_at | 0.36 | PITX1 | paired-like homeodomain 1 |
| 221875_x_at | 0.36 | HLA-F | major histocompatibility complex, class I, F |
| 201438_at | 0.359 | COL6A3 | collagen, type VI, alpha 3 |
| 201851_at | 0.359 | SH3GL1 | SH3-domain GRB2-like 1 |
| 201102_s_at | 0.358 | PFKL | phosphofructokinase, liver |
| 211527_x_at | 0.358 | VEGFA | vascular endothelial growth factor A |
| 211529_x_at | 0.358 | HLA-G | major histocompatibility complex, class I, G |
| 221274_s_at | 0.358 | LMAN2L | lectin, mannose-binding 2-like |
| 221677_s_at | 0.358 | DONSON | downstream neighbor of SON |
| 212995_x_at | 0.357 | FAM128B | family with sequence similarity 128, member B |
| 201616_s_at | 0.355 | CALD1 | caldesmon 1 |
| 205180_s_at | 0.354 | ADAM8 | ADAM metallopeptidase domain 8 |
| 212759_s_at | 0.354 | TCF7L2 | transcription factor 7-like 2 (T-cell specific, |
| | | | HMG-box) |
| 208549_x_at | 0.353 | PTMA | prothymosin, alpha (gene sequence 28) |
| 204298_s_at | 0.351 | LOX | lysyl oxidase |
| 212014_x_at | 0.347 | CD44 | CD44 molecule (Indian blood group) |
| 209081_s_at | 0.346 | COL18A1 | collagen, type XVIII, alpha 1 |
| 218321_x_at | 0.345 | STYXL1 | serine/threonine/tyrosine interacting-like 1 |
| 213166_x_at | 0.344 | FAM128A | family with sequence similarity 128, member A |
| 209193_at | 0.339 | PIM1 | pim-1 oncogene |
| 202270_at | 0.338 | GBP1 | guanylate binding protein 1, interferon-inducible, |
| | | | 67 kDa |
| 203973_s_at | 0.338 | CEBPD | CCAAT/enhancer binding protein (C/EBP), delta |
| 200739_s_at | 0.333 | SUMO3 | SMT3 suppressor of mif two 3 homolog 3 (S. cerevisiae) |
| 200824_at | 0.33 | GSTP1 | glutathione S-transferase pi |
| 211840_s_at | 0.327 | PDE4D | phosphodiesterase 4D, cAMP-specific |
| | | | (phosphodiesterase E3 dunce homolog, |
| | | | Drosophila) |
| 201615_x_at | 0.322 | CALD1 | caldesmon 1 |
| 206023_at | 0.321 | NMU | neuromedin U |
| 217478_s_at | 0.317 | HLA-DMA | major histocompatibility complex, class II, DM |
| | | | alpha |
| 208445_s_at | 0.313 | BAZ1B | bromodomain adjacent to zinc finger domain, 1B |
| 209619_at | 0.313 | CD74 | CD74 molecule, major histocompatibility |
| | | | complex, class II invariant chain |
| 202748_at | 0.31 | GBP2 | guanylate binding protein 2, interferon-inducible |
| 209140_x_at | 0.309 | HLA-B | major histocompatibility complex, class I, B |
| 57715_at | 0.309 | FAM26B | family with sequence similarity 26, member B |
| 201108_s_at | 0.307 | THBS1 | thrombospondin 1 |
| 211799_x_at | 0.307 | HLA-C | major histocompatibility complex, class I, C |
| 205490_x_at | 0.305 | GJB3 | gap junction protein, beta 3, 31 kDa |
| 206632_s_at | 0.305 | APOBEC3B | apolipoprotein B mRNA editing enzyme, |
| | | | catalytic polypeptide-like 3B |
| 207494_s_at | 0.303 | ZNF76 | zinc finger protein 76 (expressed in testis) |
| 209784_s_at | 0.303 | JAG2 | jagged 2 |
| 202269_x_at | 0.301 | GBP1 | guanylate binding protein 1, interferon-inducible, |
| | | | 67 kDa |
| 209832_s_at | 0.301 | CDT1 | chromatin licensing and DNA replication factor 1 |
| 203099_s_at | 0.296 | CDYL | chromodomain protein, Y-like |
| 203186_s_at | 0.295 | S100A4 | S100 calcium binding protein A4 |
| 219561_at | 0.291 | COPZ2 | coatomer protein complex, subunit zeta 2 |
| 204446_s_at | 0.29 | ALOX5 | arachidonate 5-lipoxygenase |
| 217517_x_at | 0.29 | SRPK2 | SFRS protein kinase 2 |
| 221039_s_at | 0.285 | DDEF1 | development and differentiation enhancing factor 1 |
| 211839_s_at | 0.284 | CSF1 | colony stimulating factor 1 (macrophage) |
| 217889_s_at | 0.284 | CYBRD1 | cytochrome b reductase 1 |
| 218018_at | 0.283 | PDXK | pyridoxal (pyridoxine, vitamin B6) kinase |
| 201368_at | 0.282 | ZFP36L2 | zinc finger protein 36, C3H type-like 2 |
| 201369_s_at | 0.276 | ZFP36L2 | zinc finger protein 36, C3H type-like 2 |
| 205179_s_at | 0.272 | ADAM8 | ADAM metallopeptidase domain 8 |
| 211911_x_at | 0.27 | HLA-B | major histocompatibility complex, class I, B |
| 209365_s_at | 0.267 | ECM1 | extracellular matrix protein 1 |
| 210514_x_at | 0.267 | HLA-G | major histocompatibility complex, class I, G |
| 203315_at | 0.264 | NCK2 | NCK adaptor protein 2 |
| 219550_at | 0.264 | ROBO3 | roundabout, axon guidance receptor, homolog 3 |
| | | | (Drosophila) |
| 219054_at | 0.262 | C5orf23 | chromosome 5 open reading frame 23 |
| 204543_at | 0.261 | RAPGEF1 | Rap guanine nucleotide exchange factor (GEF) 1 |
| 201367_s_at | 0.257 | ZFP36L2 | zinc finger protein 36, C3H type-like 2 |
| 203153_at | 0.25 | IFIT1 | interferon-induced protein with tetratricopeptide |
| | | | repeats 1 |
| 213792_s_at | 0.25 | INSR | insulin receptor |
| 222017_x_at | 0.241 | LRCH4 | leucine-rich repeats and calponin homology (CH) |
| | | | domain containing 4 |
| 214463_x_at | 0.24 | HIST1H4J | histone cluster 1, H4j |
| 204070_at | 0.237 | RARRES3 | retinoic acid receptor responder (tazarotene |
| | | | induced) 3 |
| 201508_at | 0.236 | IGFBP4 | insulin-like growth factor binding protein 4 |
| 201841_s_at | 0.226 | HSPB1 | heat shock 27 kDa protein 1 |
| 205663_at | 0.222 | PCBP3 | poly(rC) binding protein 3 |
| 209183_s_at | 0.217 | C10orf10 | chromosome 10 open reading frame 10 |
| 52975_at | 0.212 | FAM125B | family with sequence similarity 125, member B |
| 210830_s_at | 0.21 | PON2 | paraoxonase 2 |
| 204560_at | 0.209 | FKBP5 | FK506 binding protein 5 |
| 208729_x_at | 0.206 | HLA-B | major histocompatibility complex, class I, B |
| 207833_s_at | 0.205 | HLCS | holocarboxylase synthetase (biotin-(proprionyl- |
| | | | Coenzyme A-carboxylase (ATP-hydrolysing)) |
| | | | ligase) |
| 210776_x_at | 0.192 | TCF3 | transcription factor 3 (E2A immunoglobulin |
| | | | enhancer binding factors E12/E47) |
| 201107_s_at | 0.191 | THBS1 | thrombospondin 1 |
| 219594_at | 0.174 | NINJ2 | ninjurin 2 |
| 209256_s_at | 0.173 | KIAA0265 | KIAA0265 protein |
| 213075_at | 0.172 | OLFML2A | olfactomedin-like 2A |
| 201876_at | 0.171 | PON2 | paraoxonase 2 |
| 202986_at | 0.17 | ARNT2 | aryl-hydrocarbon receptor nuclear translocator 2 |
| 203770_s_at | 0.17 | STS | steroid sulfatase (microsomal), isozyme S |
| 204148_s_at | 0.17 | ZP3 | zona pellucida glycoprotein 3 (sperm receptor) |
| 209641_s_at | 0.17 | ABCC3 | ATP-binding cassette, sub-family C |
| | | | (CFTR/MRP), member 3 |
| 213724_s_at | 0.17 | PDK2 | pyruvate dehydrogenase kinase, isozyme 2 |
| 211182_x_at | 0.167 | RUNX1 | runt-related transcription factor 1 (acute myeloid |
| | | | leukemia 1; aml1 oncogene) |
| 203702_s_at | 0.163 | TTLL4 | tubulin tyrosine ligase-like family, member 4 |
| 204268_at | 0.162 | S100A2 | S100 calcium binding protein A2 |
| 218537_at | 0.161 | HCFC1R1 | host cell factor C1 regulator 1 (XPO1 dependent) |
| 219878_s_at | 0.153 | KLF13 | Kruppel-like factor 13 |
| 221565_s at | 0.151 | FAM26B | family with sequence similarity 26, member B |
| 221687_s_at | 0.142 | FAM125B | family with sequence similarity 125, member B |
| 210140_at | 0.118 | CST7 | cystatin F (leukocystatin) |
| 211372_s_at | 0.118 | IL1R2 | interleukin 1 receptor, type II |
| 202688_at | 0.116 | TNFSF10 | tumor necrosis factor (ligand) superfamily, |
| | | | member 10 |
| 220233_at | 0.0971 | FBXO17 | F-box protein 17 |
| 209616_s_at | 0.0852 | CES1 | carboxylesterase 1 (monocyte/macrophage serine |
| | | | esterase 1) |
| 203828_s_at | 0.083 | IL32 | interleukin 32 |
| 205119_s_at | 0.0751 | FPR1 | formyl peptide receptor 1 |
| 204040_at | 0.0707 | RNF144A | ring finger protein 144A |
| 201427_s_at | 0.0283 | SEPP1 | selenoprotein P, plasma, 1 |
| 206067_s_at | 0.0255 | WT1 | Wilms tumor 1 |

TABLE 4
Class comparison between parental MDA231 and its brain
metastatic derivatives. These 210 probe sets represent 179
genes differentially expressed genes by more than 2.5 fold
between the two classes. Fold change values are indicated.

| Probe set | Fold | Gene symbol | Gene name |
|---|---|---|---|
| 205563_at | 91.02 | KISS1 | KiSS-1 metastasis-suppressor |
| 204475_at | 32.81 | MMP1 | matrix metallopeptidase 1 (interstitial |
| | | | collagenase) |
| 210119_at | 21.58 | KCNJ15 | potassium inwardly-rectifying channel, |
| | | | subfamily J, member 15 |
| 201044_x_at | 19.73 | DUSP1 | dual specificity phosphatase 1 |
| 200665_s_at | 17.76 | SPARC | secreted protein, acidic, cysteine-rich |
| | | | (osteonectin) |
| 206172_at | 14.72 | IL13RA2 | interleukin 13 receptor, alpha 2 |
| 204220_at | 13.93 | GMFG | glia maturation factor, gamma |
| 207442_at | 13.55 | CSF3 | colony stimulating factor 3 (granulocyte) |
| 204933_s_at | 13.23 | TNFRSF11B | tumor necrosis factor receptor superfamily, |
| | | | member 11b (osteoprotegerin) |
| 204748_at | 11.51 | PTGS2 | prostaglandin-endoperoxide synthase 2 |
| | | | (prostaglandin G/H synthase and |
| | | | cyclooxygenase) |
| 213280_at | 10.97 | GARNL4 | GTPase activating Rap/RanGAP domain- |
| | | | like 4 |
| 205547_s_at | 10.89 | TAGLN | transgelin |
| 210310_s_at | 9.799 | FGF5 | fibroblast growth factor 5 |
| 205067_at | 9.563 | IL1B | interleukin 1, beta |
| 222162_s_at | 8.779 | ADAMTS1 | ADAM metallopeptidase with |
| | | | thrombospondin type 1 motif, 1 |
| AFFX- | 8.752 | LOC100008589 | 28S ribosomal RNA |
| M27830_M_at | | | |
| 206385_s_at | 8.356 | ANK3 | ankyrin 3, node of Ranvier (ankyrin G) |
| 204614_at | 7.792 | SERPINB2 | serpin peptidase inhibitor, clade B |
| | | | (ovalbumin), member 2 |
| 214467_at | 7.667 | GPR65 | G protein-coupled receptor 65 |
| 209833_at | 7.355 | CRADD | CASP2 and RIPK1 domain containing |
| | | | adaptor with death domain |
| 205534_at | 7.264 | PCDH7 | protocadherin 7 |
| 219271_at | 7.119 | GALNT14 | UDP-N-acetyl-alpha-D- |
| | | | galactosamine:polypeptide N- |
| | | | acetylgalactosaminyltransferase 14 |
| | | | (GalNAc-T14) |
| 215071_s_at | 6.641 | HIST1H2AC | histone cluster 1, H2ac |
| 220308_at | 6.474 | CCDC19 | coiled-coil domain containing 19 |
| 202859_x_at | 6.153 | IL8 | interleukin 8 |
| 205569_at | 6.109 | LAMP3 | lysosomal-associated membrane protein 3 |
| 201041_s_at | 6.008 | DUSP1 | dual specificity phosphatase 1 |
| 208180_s_at | 5.942 | HIST1H4H | histone cluster 1, H4h |
| 214290_s_at | 5.828 | HIST2H2AA3 | histone cluster 2, H2aa3 |
| 206432_at | 5.684 | HAS2 | hyaluronan synthase 2 |
| 201809_s_at | 5.609 | ENG | endoglin (Osler-Rendu-Weber syndrome 1) |
| 212667_at | 5.455 | SPARC | secreted protein, acidic, cysteine-rich |
| | | | (osteonectin) |
| 219274_at | 5.381 | TSPAN12 | tetraspanin 12 |
| 219815_at | 5.366 | GAL3ST4 | galactose-3-O-sulfotransferase 4 |
| 201645_at | 5.298 | TNC | tenascin C (hexabrachion) |
| 217388_s_at | 5.273 | KYNU | kynureninase (L-kynurenine hydrolase) |
| 205828_at | 5.214 | MMP3 | matrix metallopeptidase 3 (stromelysin 1, |
| | | | progelatinase) |
| 208608_s_at | 5.139 | SNTB1 | syntrophin, beta 1 (dystrophin-associated |
| | | | protein A1, 59 kDa, basic component 1) |
| 61734_at | 5.079 | RCN3 | reticulocalbin 3, EF-hand calcium binding |
| | | | domain |
| 207075_at | 5.048 | NLRP3 | NLR family, pyrin domain containing 3 |
| 201858_s_at | 4.997 | SRGN | serglycin |
| 209201_x_at | 4.981 | CXCR4 | chemokine (C—X—C motif) receptor 4 |
| 202388_at | 4.887 | RGS2 | regulator of G-protein signaling 2, 24 kDa |
| 213194_at | 4.783 | ROBO1 | roundabout, axon guidance receptor, |
| | | | homolog 1 (Drosophila) |
| 210103_s_at | 4.776 | FOXA2 | forkhead box A2 |
| 207321_s_at | 4.749 | ABCB9 | ATP-binding cassette, sub-family B |
| | | | (MDR/TAP), member 9 |
| 206280_at | 4.731 | CDH18 | cadherin 18, type 2 |
| 39402_at | 4.671 | IL1B | interleukin 1, beta |
| 213988_s_at | 4.653 | SAT1 | spermidine/spermine N1-acetyltransferase 1 |
| 204596_s_at | 4.627 | STC1 | stanniocalcin 1 |
| 208378_x_at | 4.569 | FGF5 | fibroblast growth factor 5 |
| 221009_s_at | 4.525 | ANGPTL4 | angiopoietin-like 4 |
| 219308_s_at | 4.504 | AK5 | adenylate kinase 5 |
| 214455_at | 4.489 | HIST1H2BC | histone cluster 1, H2bc |
| 202806_at | 4.455 | DBN1 | drebrin 1 |
| 211506_s_at | 4.286 | IL8 | interleukin 8 |
| 212636_at | 4.285 | QKI | quaking homolog, KH domain RNA |
| | | | binding (mouse) |
| 209911_x_at | 4.233 | HIST1H2BD | histone cluster 1, H2bd |
| 209398_at | 4.22 | HIST1H1C | histone cluster 1, H1c |
| 203083_at | 4.196 | THBS2 | thrombospondin 2 |
| 201288_at | 4.185 | ARHGDIB | Rho GDP dissociation inhibitor (GDI) beta |
| 213669_at | 4.176 | FCHO1 | FCH domain only 1 |
| 204749_at | 4.095 | NAP1L3 | nucleosome assembly protein 1-like 3 |
| 218280_x_at | 4.053 | HIST2H2AA3 | histone cluster 2, H2aa3 |
| 220483_s_at | 4.036 | RNF19A | ring finger protein 19A |
| 207463_x_at | 4.028 | PRSS3 | protease, serine, 3 (mesotrypsin) |
| 218319_at | 4.011 | PELI1 | pellino homolog 1 (Drosophila) |
| 210663_s_at | 3.966 | KYNU | kynureninase (L-kynurenine hydrolase) |
| 201808_s_at | 3.846 | ENG | endoglin (Osler-Rendu-Weber syndrome 1) |
| 207334_s_at | 3.84 | TGFBR2 | transforming growth factor, beta receptor II |
| | | | (70/80 kDa) |
| 208579_x_at | 3.795 | H2BFS | H2B histone family, member S |
| 214954_at | 3.782 | SUSD5 | sushi domain containing 5 |
| 218995_s_at | 3.733 | EDN1 | endothelin 1 |
| 209763_at | 3.715 | CHRDL1 | chordin-like 1 |
| 213181_s_at | 3.713 | MOCS1 | molybdenum cofactor synthesis 1 |
| 211919_s_at | 3.65 | CXCR4 | chemokine (C—X—C motif) receptor 4 |
| 219377_at | 3.639 | FAM59A | family with sequence similarity 59, |
| | | | member A |
| 202864_s_at | 3.629 | SP100 | SP100 nuclear antigen |
| 204298_s_at | 3.62 | LOX | lysyl oxidase |
| 218573_at | 3.575 | MAGEH1 | melanoma antigen family H, 1 |
| 205352_at | 3.539 | SERPINI1 | serpin peptidase inhibitor, clade I |
| | | | (neuroserpin), member 1 |
| 211368_s_at | 3.533 | CASP1 | caspase 1, apoptosis-related cysteine |
| | | | peptidase (interleukin 1, beta, convertase) |
| 205986_at | 3.517 | AATK | apoptosis-associated tyrosine kinase |
| 206232_s_at | 3.495 | B4GALT6 | UDP-Gal:betaGlcNAc beta 1,4- |
| | | | galactosyltransferase, polypeptide 6 |
| 215446_s_at | 3.488 | LOX | lysyl oxidase |
| 219778_at | 3.478 | ZFPM2 | zinc finger protein, multitype 2 |
| 210592_s_at | 3.475 | SAT1 | spermidine/spermine N1-acetyltransferase 1 |
| 208527_x_at | 3.453 | HIST1H2BE | histone cluster 1, H2be |
| 210307_s_at | 3.436 | KLHL25 | kelch-like 25 (Drosophila) |
| 202912_at | 3.428 | ADM | adrenomedullin |
| 218954_s_at | 3.421 | BRF2 | BRF2, subunit of RNA polymerase III |
| | | | transcription initiation factor, BRF1-like |
| 216250_s_at | 3.398 | LPXN | leupaxin |
| 201739_at | 3.395 | SGK1 | serum/glucocorticoid regulated kinase 1 |
| 222062_at | 3.326 | IL27RA | interleukin 27 receptor, alpha |
| 219523_s_at | 3.312 | ODZ3 | odz, odd Oz/ten-m homolog 3 (Drosophila) |
| 221238_at | 3.304 | NSBP1 | nucleosomal binding protein 1 |
| 204321_at | 3.259 | NEO1 | neogenin homolog 1 (chicken) |
| 218857_s_at | 3.251 | ASRGL1 | asparaginase like 1 |
| 202627_s_at | 3.25 | SERPINE1 | serpin peptidase inhibitor, clade E (nexin, |
| | | | plasminogen activator inhibitor type 1), |
| | | | member 1 |
| 218587_s_at | 3.249 | KTELC1 | KTEL (Lys-Tyr-Glu-Leu) containing 1 |
| 209101_at | 3.24 | CTGF | connective tissue growth factor |
| 212190_at | 3.238 | SERPINE2 | serpin peptidase inhibitor, clade E (nexin, |
| | | | plasminogen activator inhibitor type 1), |
| | | | member 2 |
| 218692_at | 3.178 | GOLSYN | Golgi-localized protein |
| 32032_at | 3.17 | DGCR14 | DiGeorge syndrome critical region gene 14 |
| 202728_s_at | 3.113 | LTBP1 | latent transforming growth factor beta |
| | | | binding protein 1 |
| 206654_s_at | 3.086 | POLR3G | polymerase (RNA) III (DNA directed) |
| | | | polypeptide G (32 kD) |
| 205082_s_at | 3.066 | AOX1 | aldehyde oxidase 1 |
| 203404_at | 3.048 | ARMCX2 | armadillo repeat containing, X-linked 2 |
| 221060_s_at | 3.033 | TLR4 | toll-like receptor 4 |
| 220979_s_at | 3.028 | ST6GALNAC5 | ST6 (alpha-N-acetyl-neuraminyl-2,3-beta- |
| | | | galactosyl-1,3)-N-acetylgalactosaminide |
| | | | alpha-2,6-sialyltransferase 5 |
| 203455_s_at | 3.025 | SAT1 | spermidine/spermine N1-acetyltransferase 1 |
| 33304_at | 3.025 | ISG20 | interferon stimulated exonuclease gene |
| | | | 20 kDa |
| 208523_x_at | 3.024 | HIST1H2BI | histone cluster 1, H2bi |
| 205463_s_at | 3.012 | PDGFA | platelet-derived growth factor alpha |
| | | | polypeptide |
| 220169_at | 3.009 | TMEM156 | transmembrane protein 156 |
| 205083_at | 2.978 | AOX1 | aldehyde oxidase 1 |
| 206354_at | 2.964 | SLCO1B3 | solute carrier organic anion transporter |
| | | | family, member 1B3 |
| 209199_s_at | 2.956 | MEF2C | myocyte enhancer factor 2C |
| 222067_x_at | 2.941 | HIST1H2BD | histone cluster 1, H2bd |
| 221085_at | 2.893 | TNFSF15 | tumor necrosis factor (ligand) superfamily, |
| | | | member 15 |
| 204595_s_at | 2.889 | STC1 | stanniocalcin 1 |
| 204597_x_at | 2.853 | STC1 | stanniocalcin 1 |
| 202619_s_at | 2.831 | PLOD2 | procollagen-lysine, 2-oxoglutarate 5- |
| | | | dioxygenase 2 |
| 204222_s_at | 2.803 | GLIPR1 | GLI pathogenesis-related 1 (glioma) |
| 210654_at | 2.776 | TNFRSF10D | tumor necrosis factor receptor superfamily, |
| | | | member 10d, decoy with truncated death |
| | | | domain |
| 201487_at | 2.69 | CTSC | cathepsin C |
| 214537_at | 2.64 | HIST1H1D | histone cluster 1, H1d |
| 209565_at | 2.632 | RNF113A | ring finger protein 113A |
| 206858_s_at | 2.627 | HOXC6 | homeobox C6 |
| 202620_s_at | 2.554 | PLOD2 | procollagen-lysine, 2-oxoglutarate 5- |
| | | | dioxygenase 2 |
| 201906_s_at | 0.397 | CTDSPL | CTD (carboxy-terminal domain, RNA |
| | | | polymerase II, polypeptide A) small |
| | | | phosphatase-like |
| 202642_s_at | 0.394 | TRRAP | transformation/transcription domain- |
| | | | associated protein |
| 203690_at | 0.389 | TUBGCP3 | tubulin, gamma complex associated protein 3 |
| 202481_at | 0.385 | DHRS3 | dehydrogenase/reductase (SDR family) |
| | | | member 3 |
| 203314_at | 0.385 | GTPBP6 | GTP binding protein 6 (putative) |
| 202873_at | 0.384 | ATP6V1C1 | ATPase, H+ transporting, lysosomal |
| | | | 42 kDa, V1 subunit C1 |
| 204341_at | 0.381 | TRIM16 | tripartite motif-containing 16 |
| 219038_at | 0.381 | MORC4 | MORC family CW-type zinc finger 4 |
| 204765_at | 0.374 | ARHGEF5 | Rho guanine nucleotide exchange factor |
| | | | (GEF) 5 |
| 202610_s_at | 0.373 | MED14 | mediator complex subunit 14 |
| 213275_x_at | 0.373 | CTSB | cathepsin B |
| 208824_x_at | 0.372 | PCTK1 | PCTAIRE protein kinase 1 |
| 212192_at | 0.366 | KCTD12 | potassium channel tetramerisation domain |
| | | | containing 12 |
| 36554_at | 0.365 | ASMTL | acetylserotonin O-methyltransferase-like |
| 202886_s_at | 0.36 | PPP2R1B | protein phosphatase 2 (formerly 2A), |
| | | | regulatory subunit A, beta isoform |
| 203325_s_at | 0.354 | COL5A1 | collagen, type V, alpha 1 |
| 213400_s_at | 0.354 | TBL1X | transducin (beta)-like 1X-linked |
| 211208_s_at | 0.35 | CASK | calcium/calmodulin-dependent serine |
| | | | protein kinase (MAGUK family) |
| 203640_at | 0.348 | MBNL2 | muscleblind-like 2 (Drosophila) |
| 212188_at | 0.347 | KCTD12 | potassium channel tetramerisation domain |
| | | | containing 12 |
| 213947_s_at | 0.346 | NUP210 | nucleoporin 210 kDa |
| 202275_at | 0.341 | G6PD | glucose-6-phosphate dehydrogenase |
| 207239_s_at | 0.337 | PCTK1 | PCTAIRE protein kinase 1 |
| 203767_s_at | 0.336 | STS | steroid sulfatase (microsomal), isozyme S |
| 212826_s_at | 0.328 | SLC25A6 | solute carrier family 25 (mitochondrial |
| | | | carrier; adenine nucleotide translocator), |
| | | | member 6 |
| 203974_at | 0.324 | HDHD1A | haloacid dehalogenase-like hydrolase |
| | | | domain containing 1A |
| 218717_s_at | 0.323 | LEPREL1 | leprecan-like 1 |
| 201841_s_at | 0.322 | HSPB1 | heat shock 27 kDa protein 1 |
| 219489_s_at | 0.32 | NXN | nucleoredoxin |
| 202756_s_at | 0.318 | GPC1 | glypican 1 |
| 210202_s_at | 0.314 | BIN1 | bridging integrator 1 |
| 202245_at | 0.313 | LSS | lanosterol synthase (2,3-oxidosqualene- |
| | | | lanosterol cyclase) |
| 218907_s_at | 0.31 | LRRC61 | leucine rich repeat containing 61 |
| 202853_s_at | 0.309 | RYK | RYK receptor-like tyrosine kinase |
| 201876_at | 0.308 | PON2 | paraoxonase 2 |
| 205239_at | 0.296 | AREG | amphiregulin (schwannoma-derived |
| | | | growth factor) |
| 209082_s_at | 0.293 | COL18A1 | collagen, type XVIII, alpha 1 |
| 218035_s_at | 0.293 | RBM47 | RNA binding motif protein 47 |
| 202611_s_at | 0.289 | MED14 | mediator complex subunit 14 |
| 207214_at | 0.287 | SPINK4 | serine peptidase inhibitor, Kazal type 4 |
| 205843_x_at | 0.286 | CRAT | carnitine acetyltransferase |
| 218153_at | 0.283 | CARS2 | cysteinyl-tRNA synthetase 2, |
| | | | mitochondrial (putative) |
| 210830_s_at | 0.273 | PON2 | paraoxonase 2 |
| 207620_s_at | 0.264 | CASK | calcium/calmodulin-dependent serine |
| | | | protein kinase (MAGUK family) |
| 209394_at | 0.26 | ASMTL | acetylserotonin O-methyltransferase-like |
| 219181_at | 0.256 | LIPG | lipase, endothelial |
| 203453_at | 0.253 | SCNN1A | sodium channel, nonvoltage-gated 1 alpha |
| 203490_at | 0.248 | ELF4 | E74-like factor 4 (ets domain transcription |
| | | | factor) |
| 206595_at | 0.245 | CST6 | cystatin E/M |
| 211518_s_at | 0.245 | BMP4 | bone morphogenetic protein 4 |
| 204989_s_at | 0.239 | ITGB4 | integrin, beta 4 |
| 202145_at | 0.235 | LY6E | lymphocyte antigen 6 complex, locus E |
| 208792_s_at | 0.235 | CLU | clusterin |
| 209081_s_at | 0.233 | COL18A1 | collagen, type XVIII, alpha 1 |
| 202017_at | 0.23 | EPHX1 | epoxide hydrolase 1, microsomal |
| | | | (xenobiotic) |
| 212942_s_at | 0.219 | KIAA1199 | KIAA1199 |
| 211839_s_at | 0.213 | CSF1 | colony stimulating factor 1 (macrophage) |
| 52975_at | 0.209 | FAM125B | family with sequence similarity 125, |
| | | | member B |
| 201869_s_at | 0.189 | TBL1X | transducin (beta)-like 1X-linked |
| 201428_at | 0.179 | CLDN4 | claudin 4 |
| 210762_s_at | 0.175 | DLC1 | deleted in liver cancer 1 |
| 204381_at | 0.165 | LRP3 | low density lipoprotein receptor-related |
| | | | protein 3 |
| 204990_s_at | 0.158 | ITGB4 | integrin, beta 4 |
| 202435_s_at | 0.153 | CYP1B1 | cytochrome P450, family 1, subfamily B, |
| | | | polypeptide 1 |
| 202436_s_at | 0.15 | CYP1B1 | cytochrome P450, family 1, subfamily B, |
| | | | polypeptide 1 |
| 216060_s_at | 0.15 | DAAM1 | dishevelled associated activator of |
| | | | morphogenesis 1 |
| 202437_s_at | 0.148 | CYP1B1 | cytochrome P450, family 1, subfamily B, |
| | | | polypeptide 1 |
| 204268_at | 0.148 | S100A2 | S100 calcium binding protein A2 |
| 209739_s_at | 0.143 | PNPLA4 | patatin-like phospholipase domain |
| | | | containing 4 |
| 213075_at | 0.13 | OLFML2A | olfactomedin-like 2A |
| 214827_at | 0.127 | PARD6B | par-6 partitioning defective 6 homolog beta |
| | | | (C. elegans) |
| 204070_at | 0.122 | RARRES3 | retinoic acid receptor responder (tazarotene |
| | | | induced) 3 |
| 212151_at | 0.112 | PBX1 | pre-B-cell leukemia homeobox 1 |
| 202434_s_at | 0.106 | CYP1B1 | cytochrome P450, family 1, subfamily B, |
| | | | polypeptide 1 |
| 212148_at | 0.0668 | PBX1 | Pre-B-cell leukemia homeobox 1 |
| AFFX-ThrX-5_at | 0.0633 | — | — |
| 208161_s_at | 0.0582 | ABCC3 | ATP-binding cassette, sub-family C |
| | | | (CFTR/MRP), member 3 |
| 209496_at | 0.0326 | RARRES2 | retinoic acid receptor responder (tazarotene |
| | | | induced) 2 |
| 208791_at | 0.0323 | CLU | clusterin |
| 202898_at | 0.0284 | SDC3 | syndecan 3 |

TABLE 5
Number of tumors and of brain relapses, and their distribution according
to ER status, in four cohorts of primary tumors.

| | All | | | | | |
|---|---|---|---|---|---|---|
| | tumors | | ER− tumors | | ER+ tumors | |
| | | Brain | | Brain | | Brain |
| | | relapse | | relapse | | relapse |
| Cohort | Tumors | (%) | Tumors | (%) | Tumors | (%) |
| MSK-82 | 82 | 5 (6.1) | 36 | 4 (11.1) | 46 | 1 (2.2) |
| EMC-286 | 286 | 10 (3.5) | 77 | 5 (6.5) | 209 | 5 (2.4) |
| NKI-295 | 295 | 22 (7.5) | 67 | 10 (14.9) | 228 | 12 (5.3) |
| EMC-204 | 204 | 16 (7.8) | 80 | 11 (13.8) | 124 | 5 (4.0) |

### REFERENCES

1 El Kamar, F. G. and Posner, J. B., Brain metastases. Semin Neurol. 24 (4), 347 (2004).
Lassman, A. B. and DeAngelis, L. M., Brain metastases. Neurol Clin. 21 (1), 1 (2003).
Tosoni, A., Ermani, M., and Brandes, A. A., The pathogenesis and treatment of brain metastases: a comprehensive review. Crit. Rev Oncol Hematol 52 (3), 199 (2004).
Weil, R. J. et al., Breast cancer metastasis to the central nervous system. Am J Pathol. 167 (4), 913 (2005).
Nathoo, N., Chahlavi, A., Barnett, G. H., and Toms, S. A., Pathobiology of brain metastases. J Clin Pathol. 58 (3), 237 (2005).
Minn, A. J. et al., Distinct organ-specific metastatic potential of individual breast cancer cells and primary tumors. J Clin Invest 115 (1), 44 (2005).
Cailleau, R., Young, R., Olive, M., and Reeves, W. J., Jr., Breast tumor cell lines from pleural effusions. J Natl Cancer Inst 53 (3), 661 (1974).
Kang, Y. et al., A multigenic program mediating breast cancer metastasis to bone. Cancer Cell. 3 (6), 537 (2003).
Minn, A. J. et al., Genes that mediate breast cancer metastasis to lung. Nature. 436 (7050), 518 (2005).
Kesari, S. and Batchelor, T. T., Leptomeningeal metastases. Neurol Clin 21 (1), 25 (2003).
Zhang, M. and Olsson, Y., Hematogenous metastases of the human brain—characteristics of peritumoral brain changes: a review. J Neurooncol. 35 (1), 81 (1997).
Minn, A. J. et al., Lung metastasis genes couple breast tumor size and metastatic spread. Proc Natl Acad Sci USA 104 (16), 6740 (2007).
Slimane, K. et al., Risk factors for brain relapse in patients with metastatic breast cancer. Ann Oncol 15 (11), 1640 (2004).
Gupta, G. P. et al., Mediators of vascular remodelling co-opted for sequential steps in lung metastasis. Nature 446 (7137), 765 (2007).
Egeblad, M. and Werb, Z., New functions for the matrix metalloproteinases in cancer progression. Nat Rev Cancer 2 (3), 161 (2002).
Padua, D. et al., TGFbeta primes breast tumors for lung metastasis seeding through angiopoietin-like 4. Cell 133 (1), 66 (2008).
Saharinen, J., Hyytiainen, M., Taipale, J., and Keski-Oja, J., Latent transforming growth factor-beta binding proteins (LTBPs)—structural extracellular matrix proteins for targeting TGF-beta action. Cytokine Growth Factor Rev 10 (2), 99 (1999).
Adams, J. C., Roles of fascin in cell adhesion and motility. Curr Opin Cell Biol 16 (5), 590 (2004).
DiSepio, D. et al., Identification and characterization of a retinoid-induced class II tumor suppressor/growth regulatory gene. Proc Natl Acad Sci USA 95 (25), 14811 (1998).
Eugenin, E. A. and Berman, J. W., Chemokine-dependent mechanisms of leukocyte trafficking across a model of the blood-brain barrier. Methods (San Diego, Calif. 29 (4), 351 (2003).
Goldstein, N. I. et al., Biological efficacy of a chimeric antibody to the epidermal growth factor receptor in a human tumor xenograft model. Clin Cancer Res 1 (11), 1311 (1995).
Okajima, T. et al., Molecular cloning of brain-specific GD1alpha synthase (ST6GalNAc V) containing CAG/Glutamine repeats. J Biol Chem 274 (43), 30557 (1999).
Ikehara, Y. et al., A novel glycosyltransferase with a polyglutamine repeat; a new candidate for GD1alpha synthase (ST6GalNAc V) (1). FEBS Lett 463 (1-2), 92 (1999).
Dall'Olio, F. and Chiricolo, M., Sialyltransferases in cancer. Glycoconj J 18 (11-12), 841 (2001).
Kaneko, Y., Yamamoto, H., Colley, K. J., and Moskal, J. R., Expression of Gal beta 1,4GlcNAc alpha 2,6-sialyltransferase and alpha 2,6-linked sialoglycoconjugates in normal human and rat tissues. J Histochem Cytochem 43 (9), 945 (1995).
Fidler, I. J., The pathogenesis of cancer metastasis: the ‘seed and soil’ hypothesis revisited. Nat Rev Cancer 3 (6), 453 (2003)
Gupta, G. P. and Massague, J., Cancer metastasis: building a framework. Cell 127 (4), 679 (2006)
Hynes, R. O., Metastatic potential: generic predisposition of the primary tumor or rare, metastatic variants-or both? Cell 113 (7), 821 (2003)
Scheel, C., Onder, T., Karnoub, A., and Weinberg, R. A., Adaptation versus selection: the origins of metastatic behavior. Cancer Res 67 (24), 11476 (2007).
Nguyen, D. X. and Massague, J., Genetic determinants of cancer metastasis. Nat Rev Genet. 8 (5), 341 (2007).
Kopp, H. G., Avecilla, S. T., Hooper, A. T., and Rafii, S., The bone marrow vascular niche: home of HSC differentiation and mobilization. Physiology (Bethesda) 20, 349 (2005).
Allan, S. M., Tyrrell, P. J., and Rothwell, N. J., Interleukin-1 and neuronal injury. Nat Rev Immunol. 5 (8), 629 (2005)
Moynagh, P. N., The interleukin-1 signalling pathway in astrocytes: a key contributor to inflammation in the brain. J Anat 207 (3), 265 (2005)
Ferrari, C. C. et al., Reversible demyelination, blood-brain barrier breakdown, and pronounced neutrophil recruitment induced by chronic IL-1 expression in the brain. Am J Pathol 165 (5), 1827 (2004)
Zhou, H. et al., A requirement for microglial TLR4 in leukocyte recruitment into brain in response to lipopolysaccharide. J Immunol 177 (11), 8103 (2006).
Mendes, O., Kim, H. T., and Stoica, G., Expression of MMP2, MMP9 and MMP3 in breast cancer brain metastasis in a rat model. Clin Exp Metastasis 22 (3), 237 (2005)
Sternlicht, M. D. et al., The stromal proteinase MMP3/stromelysin-1 promotes mammary carcinogenesis. Cell 98 (2), 137 (1999).
Wang, Y. et al., Mesotrypsin, a brain trypsin, activates selectively proteinase-activated receptor-1, but not proteinase-activated receptor-2, in rat astrocytes. J Neurochem 99 (3), 759 (2006).
Giau, R., Carrette, J., Bockaert, J., and Homburger, V., Constitutive secretion of protease nexin-1 by glial cells and its regulation by G-protein-coupled receptors. J Neurosci 25 (39), 8995 (2005).
Vaillant, C. et al., Protease nexin 1 and its receptor LRP modulate SHH signalling during cerebellar development. Development 134 (9), 1745 (2007).
Osterwalder, T. et al., Neuroserpin, an axonally secreted serine protease inhibitor. EMBO J. 15 (12), 2944 (1996); Miranda, E. and Lomas, D. A., Neuroserpin: a serpin to think about. Cell Mol Life Sci 63 (6), 709 (2006).
Gomis, R. R. et al., C/EBPbeta at the core of the TGFbeta cytostatic response and its evasion in metastatic breast cancer cells. Cancer Cell 10 (3), 203 (2006).
Ponomarev, V. et al., A novel triple-modality reporter gene for whole-body fluorescent, bioluminescent, and nuclear noninvasive imaging. Eur J Nucl Med Mol Imaging 31 (5), 740 (2004).
Cheadle, C., Cho-Chung, Y. S., Becker, K. G., and Vawter, M. P., Application of z-score transformation to Affymetrix data. Appl Bioinformatics 2 (4), 209 (2003).
Rhodes, D. R. et al., ONCOMINE: a cancer microarray database and integrated data-mining platform. Neoplasia 6 (1), 1 (2004).
Shyamsundar, R. et al., A DNA microarray survey of gene expression in normal human tissues. Genome Biol 6 (3), R22 (2005).

All of the references, patents and patents applications referred to herein are incorporated herein by reference.

What is claimed is:

1. A method of predicting the likelihood of brain metastases in a cancer patient, comprising determining the expression level of a plurality of genes/proteins from Table 1 in a sample from the cancer patient, and from the determination of expression levels predicting the likelihood of brain metastases in the patient, wherein overexpression of ANGPTL4, PLOD2, COL13A1, PTGS2, PELI1, MMP1, B4GALT6, HBEGF, CSF3, RGC32, LTBP1, FSCN1, and LAMA4 and underexpression of TNFSF10, RARRES3, SCNN1A and SEPP1 are indicative of an increased likelihood of brain metastases.

2. The method of claim 1, wherein the expression levels of all 17 genes/proteins are determined.

3. The method of claim 2, wherein the cancer patient suffers from breast cancer.

4. The method of claim 1, wherein the expression levels of at least 5 genes/proteins are determined

5. The method of claim 1, wherein the cancer patient suffers from breast cancer.

6. A kit for evaluation of cancer cells for risk of brain metastases, said kit comprising in a packaged combination specific reagents for determining the expression levels, wherein the specific reagents consist of reagents for determining a plurality of genes/proteins listed in Table 1.

7. The kit of claim 6, wherein the kit contains specific reagents for at least 5 of the genes/proteins listed in Table 1.

8. The kit of claim 6, wherein the kit contains specific reagents for all of the genes/proteins listed in Table 1.

9. A method for treating brain metastasis in a patient in need of such treatment comprising the steps of
(a) determining the expression level of a plurality of genes from Table 1 in a sample from the patient;
(b) identifying from the determination of expression levels a therapeutic targets from among the genes tested which are differentially expressed from a control value, and
(c) administering to the patient a therapeutic composition effective to normalize the level of the therapeutic target, wherein the therapeutic composition increases expression of the therapeutic target if the target is TNFSF10, RARRES3, SCNN1A or SEPP1, and the therapeutic composition decreases expression of the therapeutic target if it is ANGPTL4, PLOD2, COL13A1, PTGS2, PELI1, MMP1, B4GALT6, HBEGF, CSF3, RGC32, LTBP1, FSCN1, or LAMA4.

10. The method of claim 9, wherein the expression level of all 17 genes from Table 1 is determined.

11. The method of claim 10, wherein the patient suffers from breast cancer.

12. The method of claim 9, wherein the expression level of at least 5 genes/proteins is determined

13. The method of claim 9, wherein the patient suffers from breast cancer.

14. The method of claim 9, wherein expression levels of COX2 and HBEGF are determined.

15. A method of predicting the likelihood of brain metastases in a cancer patient, comprising determining the expression level of a plurality of genes from Table 2 in a sample from the cancer patient, and from the determination of expression levels predicting the likelihood of brain metastases in the patient, wherein overexpression of one or more of the genes is indicative of an increased likelihood of brain metastases.

16. The method of claim 15, wherein the expression level of all 18 genes is determined.

17. The method of claim 16, wherein the cancer patient suffers from breast cancer.

18. The method of claim 15, wherein the expression level of at least 5 genes/proteins is determined

19. The method of claim 15, wherein the cancer patient suffers from breast cancer.

20. A kit for evaluation of cancer cells for risk of brain metastases, said kit comprising in a packaged combination specific reagents for determining the expression levels, wherein the specific reagents consist of reagents for determining a plurality of genes/proteins listed in Table 2.

21. The kit of claim 20, wherein the kit contains specific reagents for at least 5 of the genes/proteins listed in Table 2.

22. The kit of claim 20, wherein the kit contains specific reagents for all of the genes/proteins listed in Table 2.

23. A method for treating brain metastasis in a patient in need of such treatment comprising the steps of
(a) determining the expression level of a plurality of genes from Table 2 in a sample from the patient;
(b) identifying from the determination of expression levels one or more therapeutic targets from among the genes tested which are differentially expressed from a control value, and
(c) administering to the patient a therapeutic composition effective to normalize the level of the one or more therapeutic targets, wherein the therapeutic composition decreases expression of the therapeutic target.

24. The method of claim 23, wherein the expression level of all 18 genes from table 2 is determined.

25. The method of claim 24, wherein the patient suffers from breast cancer.

26. The method of claim 23, wherein the expression level of at least 5 genes/proteins is determined

27. The method of claim 23, wherein the patient suffers from breast cancer.

28. The method of claim 23, wherein expression levels of ST6GALNAC5 is determined.

29. The method of claim 23, wherein the determined level of ST6GALNAC5 is elevated, and wherein the therapeutic composition inhibits expression of ST6GALNAC5.

30. The method of claim 29, wherein the therapeutic composition comprises an shRNA that targets ST6GALNAC5.

31. The method of claim 30, wherein the shRNA is Seq. ID No. 1 or Seq. ID No.2.
